(19) United States
(12) Patent Application Publication
(10) Pub. No.: US 2021/0196199 A1
(43) Pub. Date: Jul. 1, 2021

(54) Wireless Resonant Circuit and Variable Inductance Vascular Monitoring Implants and Anchoring Structures Therefore

(71) Applicant: Foundry Innovation & Research 1, Ltd., Dublin (IE)

(72) Inventors: Sean Quinlan, Dublin (IE); Fiachra M. Sweeney, Dublin (IE); Stephen Sheridan, Co Cavan (IE); Friedrich Wetterling, Dublin (IE)

(21) Appl. No.: 17/058,275

(22) PCT Filed: May 30, 2019
(86) PCT No.: PCT/US2019/034657

§ 371 Date: Nov. 24, 2020

Related U.S. Application Data
(60) Provisional application No. 62/678,237, filed on May 30, 2018.

(51) Int. Cl. A61B 5/00; A61B 5/07

(52) U.S. Cl. CPC A61B 5/686; A61B 5/026; A61B 5/6886; A61B 5/076

(57) ABSTRACT

Wireless, variable inductance and resonant circuit-based vascular monitoring devices, systems, methodologies, and techniques, including specifically configured anchoring structures for same, are disclosed that can be used to assist healthcare professionals in predicting, preventing, and diagnosing various heart-related and other health conditions.

### FIELD OF THE DISCLOSURE

The present invention generally relates to the field of vascular monitoring. In particular, the present invention is directed to wireless vascular monitoring implants and anchoring structures therefore. More specifically, embodiments disclosed herein relate to fluid volume sensing in the inferior vena cava (IVC) using wireless, remotely or automatically actuatable implants for monitoring or management of blood volume.

### BACKGROUND

Others have attempted to develop vascular monitoring devices and techniques, including those directed at monitoring vessel arterial or venous pressure or vessel lumen dimensions. However, many such existing systems are catheter based (not wireless) and thus can only be utilized in a clinical setting for limited periods of times, and may carry risks associated with extended catheterization. For a wireless solution, the complexity of deployment, fixation and the interrelationship of those factors with detection and communication have led to, at best, inconsistent results with such previously developed devices and techniques.

Existing wireless systems focus on pressure measurements, which in the IVC can be less responsive to patient fluid state than IVC dimension measurements. However, systems designed to measure vessel dimensions also have a number of drawbacks with respect to monitoring in the IVC. Electrical impedance-based systems require electrodes that are specifically placed in opposition across the width of the vessel. Such devices present special difficulties when attempting to monitor IVC dimensions due to the fact that the IVC does not expand and contract symmetrically as do most other vessels where monitoring may be desired. Precise positioning of such position-dependent sensors is a problem that has not yet been adequately addressed. IVC monitoring presents a further challenge arising from the physiology of the IVC. The IVC wall is relatively compliant compared to other vessels and thus can be more easily distorted by forces applied by implants to maintain their position within the vessel. Thus devices that may perform satisfactorily in other vessels may not necessarily be capable of precise monitoring in the IVC due to distortions created by force of the implant acting on the IVC wall. As such, new developments in this field are desirable in order to provide doctors and patients with reliable and affordable wireless vascular monitoring implementation, particularly in the critical area of heart failure monitoring.

### SUMMARY OF THE DISCLOSURE

Embodiments disclosed herein comprise wireless vascular monitoring devices, circuits, methodologies, and related techniques for use in assisting healthcare professionals in predicting, preventing, and diagnosing various conditions whose indicators may include vascular fluid status. Using embodiments disclosed, metrics including, for example, relative fluid status, fluid responsiveness, fluid tolerance, or heart rate may be accurately estimated.

In one implementation, the present disclosure is directed to a wireless vascular monitoring implant adapted to be deployed and implanted in a patient vasculature and positioned at a monitoring location in a vascular lumen in contact with the lumen wall. The implant includes a resilient sensor construct configured to dimensionally expand and contract with natural movement of the lumen wall; wherein an electrical property of the resilient sensor construct changes in a known relationship to the dimensional expansion and contraction thereof; and the resilient sensor construct produces a wireless signal indicative of the electrical property, the signal being readable wirelessly outside the vascular lumen to determine a dimension of the vascular lumen; the resilient sensor construct is configured and dimensioned to engage and substantially permanently implant itself on or in the lumen wall; the resilient sensor construct has a variable inductance correlated to its dimensional expansion and contraction along at least one dimension; and the resilient sensor construct produces, when energized by an energy source directed at the construct, a signal readable wirelessly outside the patient's body indicative of the value of the at least one dimension, whereby a dimension of the vascular lumen may be determined; wherein the resilient sensor construct comprises a coil configured to engage at least two opposed points on the vascular lumen wall, the coil having an inductance that varies based on the distance between the two opposed points on the coil corresponding a distance between the points on the lumen wall; wherein the coil is rotationally symmetrical about a longitudinal axis; wherein the resilient sensor construct is configured to expand and contract with the lumen wall along substantially any transverse axis of the vessel to change the variable inductance; wherein the resilient sensor construct, further comprises a frame having at least one resilient portion formed with at least two points configured to be positioned opposite one another so as to engage opposed surfaces of the vascular lumen wall when the sensor construct is positioned at the monitoring location in contact with the lumen wall, wherein the coil is formed on the frame by at least one wire disposed around the frame so as to form plural adjacent wire strands around the frame; wherein the resilient sensor construct comprises a resonant circuit having a resonant frequency that varies with the variable inductance, the signal being correlated with the resonant frequency; wherein the coil comprises a resonant circuit having inductance and a capacitance defining a resonant frequency, wherein the resonant frequency varies based on the distance between the at least two points; and the coil is configured to be energized by a magnetic field directed at the coil from outside the patient's body.

These and other aspects and features of non-limiting embodiments of the present disclosure will become apparent to those skilled in the art upon review of the following description of specific non-limiting embodiments of the invention in conjunction with the accompanying drawings.

### BRIEF DESCRIPTION OF THE DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 schematically depicts an embodiment of a wireless resonant circuit-based vascular monitoring (“RC-WVM”) system of the present disclosure;

FIG. 1A schematically depicts a portion of an alternative embodiment of a RC-WVM system of the present disclosure;

FIGS. 2 and 2A illustrate alternative embodiments of RC-WVM implants made in accordance with the teachings of the present disclosure;

FIG. 2B is a schematic, detailed view of the capacitor section of the RC-WVM implant illustrated in FIG. 2;

FIGS. 3, 3A, 3B, 3C and 3D illustrate an embodiment of a belt antenna as depicted schematically in the system of FIG. 1;

FIG. 3E schematically depicts the orientation of the antenna belt and magnetic field generated thereby with respect to an implanted RC-WVM implant;

FIG. 4 is a block diagram illustrating an embodiment of system electronics;

FIGS. 5A and 5B illustrate fixed frequency RF burst excitation signal wave forms;

FIGS. 6A and 6B illustrate sweep frequency RF burst excitation signal wave forms;

FIGS. 7A and 7B illustrate multi-frequency RF burst excitation signal wave forms;

FIG. 8 illustrates waveform pulse shaping;

FIG. 9 schematically illustrates aspects of an embodiment of a delivery system for RC-WVM implants as disclosed herein;

FIG. 9A schematically illustrates the distal end of an alternative embodiment of a delivery system for an alternative RC-WVM implant with an attached anchor frame as disclosed herein;

FIGS. 10A, 10B, 10C, 10D and 10E illustrate signals obtained in pre-clinical experiments using a prototype system and an RC-WVM implant as shown in FIGS. 1 and 2;

FIGS. 11A, 11B and 11C illustrate a further alternative RC-WVM implant embodiment in accordance with the teachings of the present disclosure;

FIG. 12 illustrates assembly of an alternative RC-WVM implant embodiment such as shown in FIGS. 11A-C;

FIG. 13 is a detailed view of an anchor structure mounted on an implant prior to encapsulation;

FIGS. 14A, 14B and 14C illustrate an alternative anchor structure for use with RC-WVM implant embodiments;

FIGS. 15A and 15B illustrate an alternative embodiment of a belt antenna for use with RC-WVM implants and systems as described herein;

FIGS. 16A and 16B illustrate recapture features to facilitate positioning and repositioning of RC-WVM implants during placement using a delivery catheter as disclosed herein;

FIG. 17 is a perspective view of an alternative RC-WVM implant embodiment with an attached anchor frame and axial anchor barbs;

FIG. 18 is a perspective view of an anchor frame as shown, for example in FIG. 17;

FIG. 19 is a detail view showing attachment of an anchor frame to a strut section of a RC-WVM implant;

FIG. 20 is a detail view showing a split in the anchor frame to prevent magnetic field coupling with the anchor frame;

FIG. 21 illustrates a further alternative embodiment in which anchor frames are disposed on both ends of a RC-WVM implant;

FIGS. 22A, 22B and 22C illustrate another embodiment of an anchor frame with anchor barbs oriented parallel to the anchor frame struts;

FIGS. 23A, 23B and 23C illustrate another embodiment of an anchor frame with anchor barbs oriented in the direction of flow in the vessel in which the RC-WVM is implanted;

FIGS. 24A and 24B illustrate yet another embodiment of an anchor frame with anchor barbs positioned at the crowns of the anchor frames;

FIG. 25A illustrates a shape set anchor frame with adjacent anchor barbs on the same side of the frame strut, and FIG. 25B shows an alternative with double anchors at each anchor location;

FIGS. 26A, 26B, 26C, 26D, 26E, 26F, 26G and 26H each illustrate alternative embodiments of anchor barbs;

FIG. 27 is a schematic cross-section showing a non-conducting connection of two anchor frame parts;

FIG. 28 shows a perspective view of a further alternative anchor frame embodiment; and

FIGS. 29A, 29B, 29C and 29D each show different alternative embodiments of anchor frame attachment arms.

### DETAILED DESCRIPTION

Aspects of the present disclosure are directed to wireless, resonant circuit-based vascular monitoring (“RC-WVM”) implants, systems, methods, and software, including excitation and feedback monitoring (“EFM”) circuits that can be used to energize an RC-WVM implant with an excitation signal and receive characteristic feedback signals produced by the RC-WVM implant. By automatically or manually analyzing the feedback produced by the RC-WVM implant, it is possible to assist healthcare professionals in predicting, preventing, and diagnosing various heart-related, kidney-related, or vascular-related health conditions. For example, the feedback produced by the RC-WVM implant at a particular time can be compared to feedback produced by the RC-WVM implant at other times and/or feedback produced by a baseline RC-WVM implant in order to understand vessel geometry and therefore estimate relative fluid status, fluid responsiveness, fluid tolerance, heart rate, respiration rate and/or other metrics. One or more of these estimations can be generated automatically or manually in order to monitor the status of a patient and provide feedback to a healthcare professional and/or the patient in case of any anomalies or relevant trends.

### System Overview

The unique physiology of the IVC presents some distinctive challenges in attempting to detect and interpret changes in its dimensions arising from changes in patient fluid state. For example, the IVC wall in a typical monitoring region (i.e., between the hepatic and renal veins) is relatively compliant compared to other vessels, which means that changes in vessel volume can result in different relative distance changes between the anterior-posterior walls as compared to the lateral-medial walls. Thus, it is quite typical that changes in fluid volume will lead to paradoxical changes in the geometry and motion of the vessel; that is, as the blood volume reduces, the IVC tends to get smaller and collapses with respiration, and as the blood volume increases, the IVC tends to get larger and the collapse with respiration is reduced. Systems and implants disclosed herein are uniquely configured to compensate for and interpret such paradoxical changes.

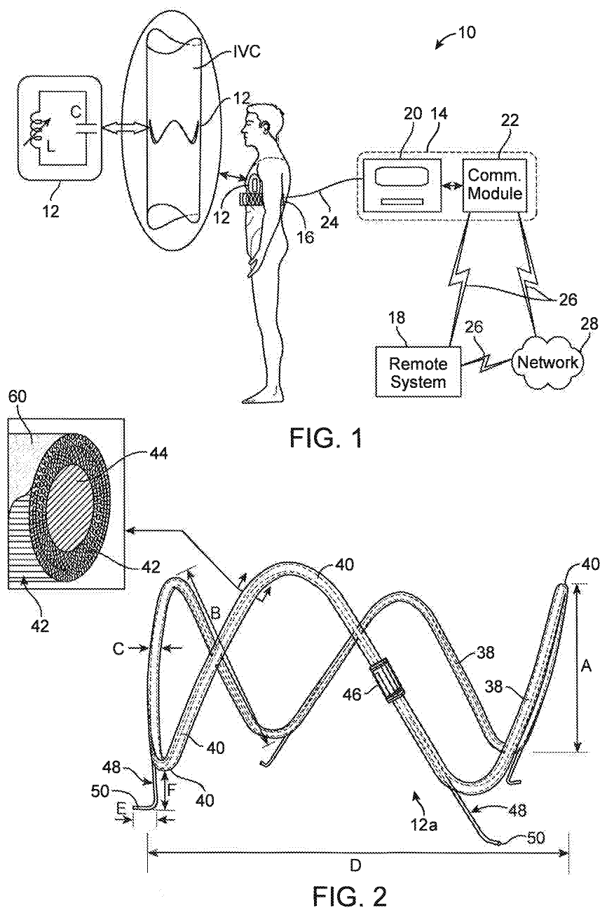

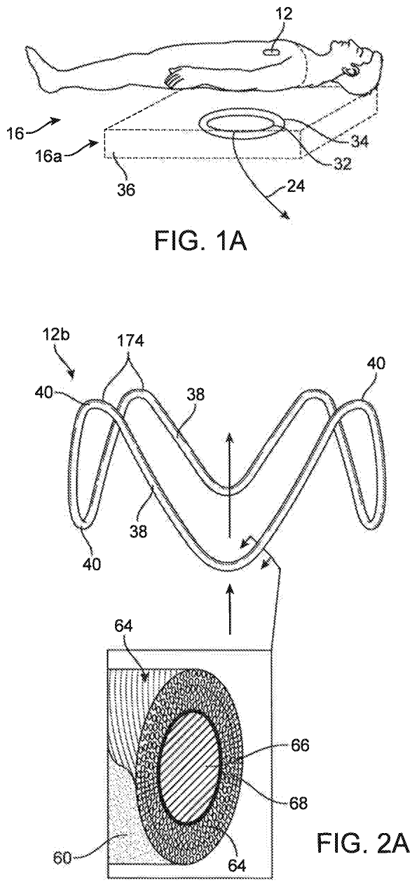

As shown in FIG. 1, system 10 according to the present disclosure may generally comprise RC-WVM implant 12 configured for placement in a patient's IVC, control system 14, antenna module 16 and one or more remote systems 18 such as processing systems, user interface/displays, data storage, etc., communicating with the control and communications modules through one or more data links 26, which may be wired or remote/wireless data links. In many implementations, remote system 18 may comprise a computing device and user interface, such as a laptop, tablet or smart phone, which serves as an external interface device.

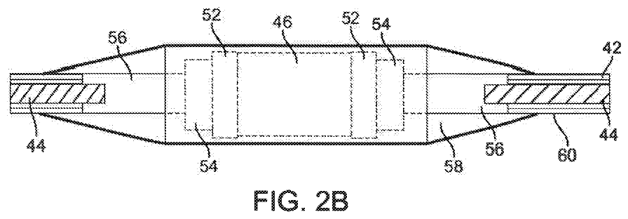

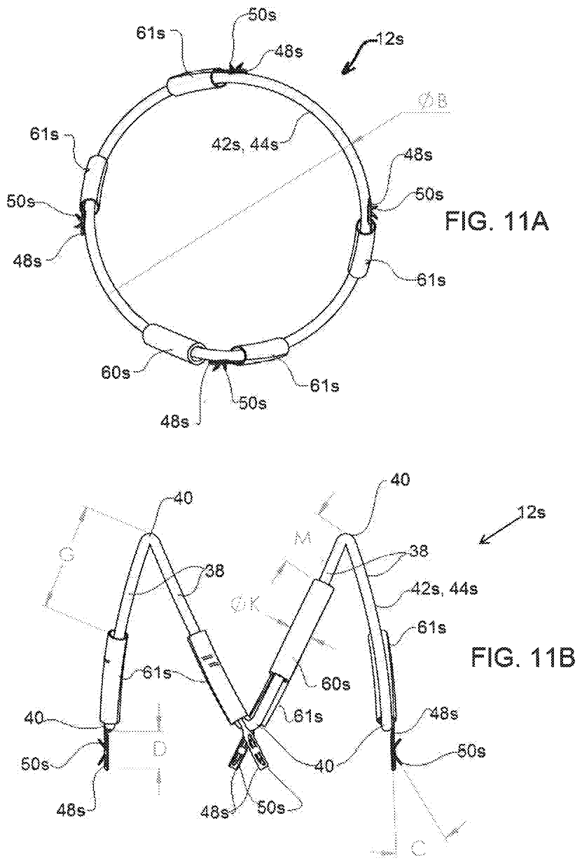

RC-WVM implant 12 generally comprises a variable inductance, constant capacitance, resonant L-C circuit formed as a resiliently collapsible coil structure, which, when positioned at a monitoring position within the patient's IVC, moves with the IVC wall as it expands and contracts due to changes in fluid volume. The variable inductance is provided by the coil structure of the implant such that the inductance changes when the dimensions of the coil change with the IVC wall movement. The capacitive element of the circuit may be provided by a discrete capacitor or specifically designed inherent capacitance of the implant structure itself. Embodiments of RC-WVM implant 12 also may be provided with anchoring and isolation means inherently designed into the implant structure, or with distinct additional such structures, to ensure that the implant is securely and properly positioned in the IVC without unduly distorting the vessel wall so as to distort or otherwise negatively impact measurements determined by the implant. In general, RC-WVM implants 12 are configured to at least substantially permanently implant themselves in the vascular lumen wall where placed upon deployment and do not require a physical connection (for communications, power or otherwise) to devices outside the patient's body after implantation. “Substantially permanently implanted” as used herein means that in normal usage the implant will, throughout its useful, operational life, remain implanted in the vascular lumen wall and may to varying degrees become integrated into the vascular lumen wall by tissue ingrowth, but the implant may be intentionally removed as medically dictated by an intravascular interventional or surgical removal procedure specifically undertaken for the purpose of removing the implant. Details of alternative embodiments of implant 12, shown in FIGS. 2, 2A, and FIGS. 11A-C, are provided below. In particular, it should be noted that any of alternative RC-WVM implants described herein may be utilized in alternative systems 10 as described herein without further modification of the systems except as may be identified.

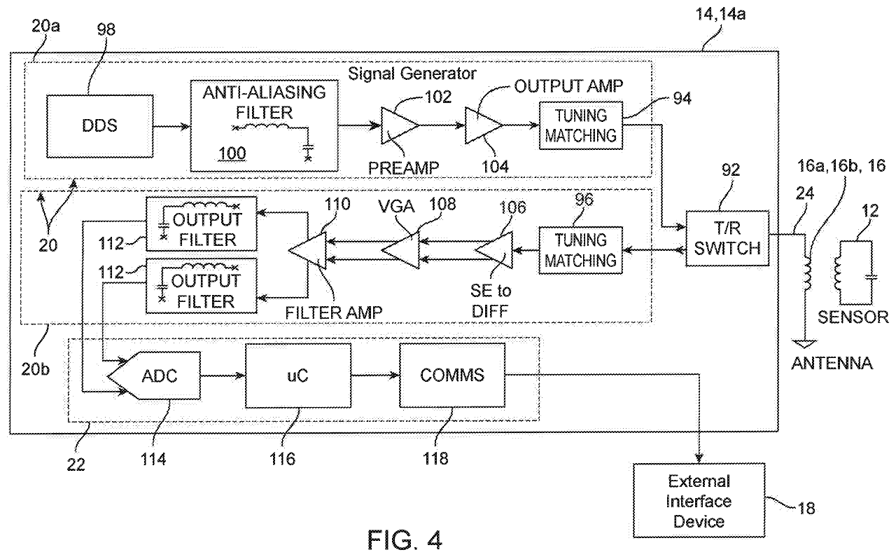

Control system 14 comprises, for example, functional modules for signal generation, signal processing and power supply (generally comprising the EFM circuits and indicated as module 20) and communications module 22 to facilitate communication and data transfer to various remote systems 18 through data links 26 and optionally other local or cloud-based networks 28. Details of an exemplary embodiment of control system 14, modules 20 and 22, and elements of alternative EFM circuits are described below and illustrated in FIG. 4. After analyzing signals received from RC-WVM implant 12 after being excited by a transmit coil of an EFM circuit, results may be communicated manually or automatically through remote system 18 to the patient, a caregiver, a medical professional, a health insurance company, and/or any other desired and authorized parties in any suitable fashion (e.g., verbally, by printing out a report, by sending a text message or e-mail, or otherwise).

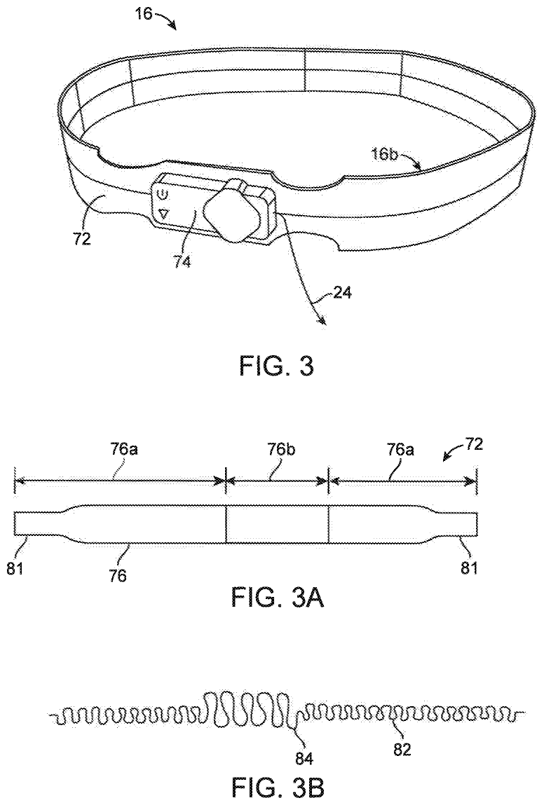

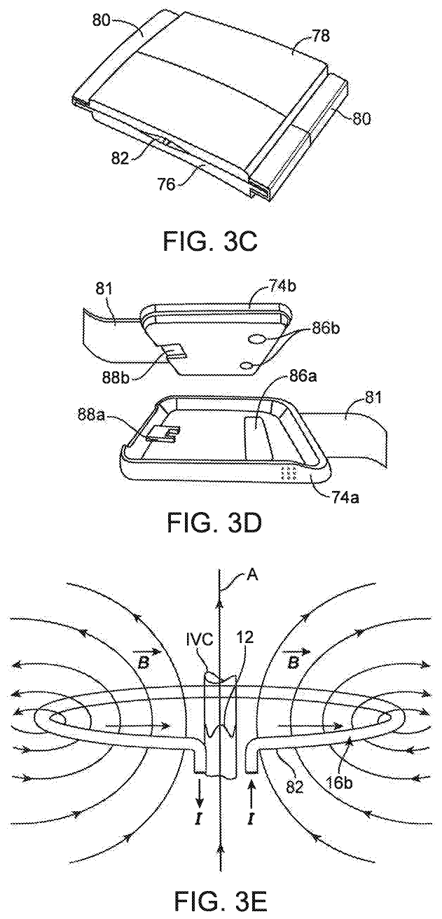

Antenna module 16 is connected to control system 14 by power and communication link 24, which may be a wired or wireless connection. Antenna module 16 creates an appropriately shaped and oriented magnetic field around RC-WVM implant 12 based on signals provided by the EFM circuitry of control system 14. The magnetic field energizes the L-C circuit of RC-WVM implant 12 causing it to produce a “ring-back” signal indicative of its inductance value at that moment. Because the inductance value is dependent on the geometry of the implant, which changes as mentioned above based on dimensional changes of the IVC in response to fluid state heart rate etc., the ring-back signal can be interpreted by control system 14 to provide information as to the IVC geometry and therefore fluid state. Antenna module 16 thus also provides a receive function/antenna as well as a transmit function/antenna. In some embodiments the transmit and receive functionality are performed by a single antenna, in others each function is performed by a separate antenna. Antenna module 16 is schematically depicted in FIG. 1 as an antenna belt, which embodiment is described in more detail below and shown in FIGS. 3A-D.

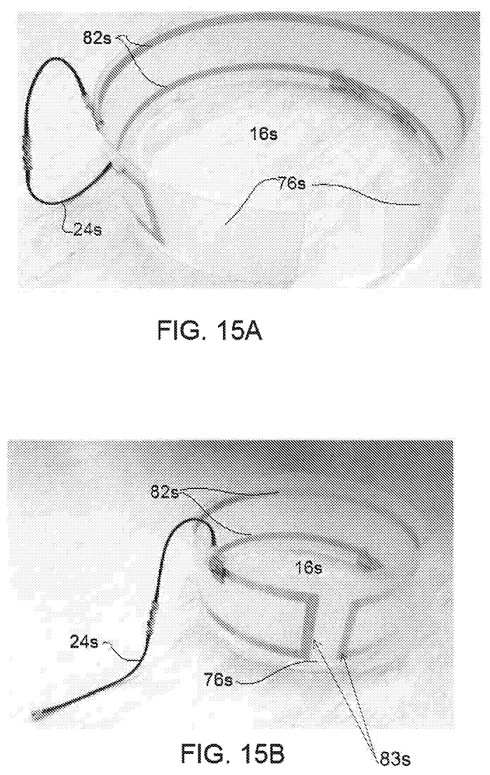

FIG. 1A illustrates one alternative embodiment of antenna module 16 as antenna pad 16a, in which transmit coil 32 and receive coil 34 are disposed in a pad or mattress 36 on which the patient lays on his/her back with RC-WVM implant 12 (implanted in the IVC) positioned over coils 32, 34. Antenna module 16 as shown in FIG. 1A is functionally equivalent to other alternative antenna modules disclosed herein; it is connected to control system 14 by power and communications link 24 as described above. Another alternative embodiment of a belt antenna module is shown in FIGS. 15A and 15B. Planar-type antenna modules also may be configured in wearable configurations, e.g., wherein the antenna coil is integrated into a wearable garment such as a backpack or vest. Antenna module 16 may also comprise a coil adapted to be fastened directly to the patient's skin by tape, glue or other means, e.g. over the abdomen or back, or integrated into furniture such as a chair back. As will be appreciated by persons skilled in the art, the various embodiments of antenna module 16 as described herein may be employed with system 10 as shown in FIG. 1 without further changes to the system or antenna module other than as specifically identified herein.

The variable inductance L-C circuit produces a resonant frequency that varies as the inductance is varied. With the implant securely fixed at a known monitoring position in the IVC, changes in geometry or dimension of the IVC cause a change in configuration of the variable inductor, which in turn cause changes in the resonant frequency of the circuit. These changes in the resonant frequency can be correlated to changes in the vessel geometry or dimension by the RC-WVM control and communication system. Thus, not only should the implant be securely positioned at a monitoring position, but also, at least a variable coil/inductor portion of the implant should have a predetermined resilience and geometry. Thus, in general, the variable inductor is specifically configured to change shape and inductance in proportion to a change in the vessel geometry. In some embodiments, an anchoring and isolation means will comprise appropriately selected and configured shape and compliance in the sensor coil structure of the implant so as to move with the vessel wall while maintaining position. Such embodiments may or may not include additional anchoring features as discussed in more detail below. Alternatively, an anchoring and isolation means may comprise a separate structure spaced and/or mechanically isolated from a variable inductor coil structure such that the anchoring function is physically and/or functionally separated from the measuring/monitoring function such that any distortion or constraint on the vessel caused by the anchor is sufficiently distant and/or isolated from the variable inductor so as not to unduly affect measurements.

RC-WVM implant 12 as a variable inductor is configured to be remotely energized by an electric field delivered by one or more transmit coils within the antenna module positioned external to the patient. When energized, the L-C circuit produces a resonant frequency which is then detected by one or more receive coils of the antenna module. Because the resonant frequency is dependent upon the inductance of the variable inductor, changes in geometry or dimension of the inductor caused by changes in geometry or dimension of the vessel wall cause changes in the resonant frequency. The detected resonant frequency is then analyzed by the RC-WVM control and communication system to determine the change in the vessel geometry or dimension. Information derived from the detected resonant frequency is processed by various signal processing techniques as described herein and may be transmitted to various remote devices such as a healthcare provider system or patient system to provide status, or in appropriate instances, alerts or modifications in treatment. In order to facilitate measurement of the detected resonant frequency, it may be desirable to provide designs with a relatively higher Q factor, i.e. resonant circuit configurations that maintain signal/energy for relatively longer periods, especially when operating at lower frequencies. For example, to realize advantages of designs employing Litz wire as further described herein, it may be desirable to operate in a resonant frequency range of below 5 MHz, typically between about 1 MHz and 3 MHz, in which case resonant circuit configuration with a Q factor of at least about 50 or greater may be desired.

### An Example of a Complete System Embodiment

Details of one possible embodiment of a complete, exemplary system 10 are discussed hereinafter with reference to FIGS. 2-8. Thereafter, details of further alternative embodiments of system components are described. However, it is to be understood that the exemplary system is not limited to use of the specific elements or components shown in FIGS. 1-8 and that any alternative component thereafter described may be substituted without change in the overall system except as may be noted.

FIG. 2 illustrates one example of RC-WVM implant 12 according to the present disclosure as may be used in exemplary system 10. The enlarged detail in the box of FIG. 2 represents a cross-sectional view taken as indicated. (Note that in the cross-sectional view, individual ends of the very fine wires may not be distinctly visible due to their very small size). In general, RC-WVM implants 12 comprise a resilient sensor construct generally including an inductive coil formed around an open center to allow substantially unimpeded blood flow there through, wherein the inductive coil changes inductance with changes in the construct geometry as a result of forces applied to it. In this example, implant 12a is formed as a resilient, concentric zig-zag or linked “Z-shapes” structure with a series of strut sections 38 joined at their ends by rounded crown sections 40 forming acute angles. The resultant structure may also be considered to be sinusoidal in appearance. This structure may be formed by wrapping conductive wires 42 onto a frame or core 44. In this alternative, RC-WVM implant 12a has a shape set 0.010″ nitinol wire frame 44 around which 300 strands of 0.04 mm diameter gold, individually insulated, Litz wire 42 are wrapped in a single loop. With a single loop wrap, the strands of wire 42 appear substantially parallel to the frame at any given point, as can be seen in the cross-sectional view of FIG. 2. Individual insulation on Litz wires 42 may be formed as a biocompatible polyurethane coating. Also in this particular example, discrete capacitor 46 is provided with a capacitance of approximately 47ηF (nano-Farads); however, the capacitance may be in the range of about 180 pico-Farads to about 10 micro-Farads, to cover all potential allowable frequency bands (from about 148.5 kHz to about 37.5 MHz) for RC-WVM implants 12.

In one alternative, rather than a relatively large number of wire strands in a single loop, a relatively few number of strands, e.g. in the range of about 10-20 strands, or more particularly about 15 strands, may be arranged in a relatively larger number of loops, e.g. in the range of about 15-25 loops, or more particularly about 20 loops. In this alternative embodiment the discrete capacitor element is replaced with an inherent coil capacitance that arises based on spaces between the parallel strands of wire.

In a further alternative embodiment, implant 12a is configured to ensure strut sections 38 are straight strut sections between crown sections 40. Straight strut sections can provide an advantage of the strut section always being in contact with the vessel wall over its entire length, irrespective of the size of vessel into which it is deployed. When the sensor construct frame is formed, for example, by laser cutting the construct from a nitinol tube, the straight configuration of the straight strut sections can be achieved by shape-setting the strut sections to maintain the desired straight configuration.

With reference also to FIG. 2B, Litz wire 42 is formed around a shape set nitinol frame 44. The two ends of Litz wire 42, which may be covered with a layer of PET heat shrink tubing 60, are joined together with a capacitor 46 to form a loop circuit. Capacitor 46 includes capacitor terminals 52 connected to Litz wires 42 by solder connection 54 to gold wire contacts 56. Gold wire contacts 56 are formed by removing (or burning away) the individual insulation from a short section at the end of Litz wires 42 and joining those ends to form solid contacts, which then may be joined to capacitor terminals 52 by solder connections 54. The capacitor, capacitor terminals and gold wire contacts are encapsulated in an appropriate biocompatible insulating material 58 such as a reflowed polymer or epoxy. In alternative embodiments, the entire structure may then be covered by a layer of PET heat shrink insulation 60. Alternatively, if determined that a short circuit through the frame should not be created, a gap may be provided in the frame at the capacitor or elsewhere.

As shown in FIG. 2, RC-WVM implant 12a is also optionally provided with anchors 48 to help prevent migration of the implant after placement in the IVC. Anchors 48 also may be formed of nitinol laser cut sections or shape set wire and bonded to each strut section 38. Barbs 50 extend outwardly at the end of anchors 48 to engage the IVC wall. In one embodiment, anchors 48 are bi-directional in both the cranial and caudal directions; in other embodiments the anchors may be in one direction, a mixture of both directions or perpendicular to the vessel.

The overall structure of RC-WVM implants 12 presents a balance of electrical and mechanical requirements. For example, an ideal electrical sensor is as close to a solenoid as possible with strut lengths as short as possible and ideally zero, whereas mechanical considerations of deployment and stability dictate that implant strut lengths be at least as long as the diameter of the vessel into which it is to be deployed to avoid deployment in the wrong orientation and maintain stability. Dimensions of elements of RC-WVM implant 12a are identified by letters A-F in FIG. 2, and examples of typical values for those dimensions, suited for a range of patient anatomies, are provided below in Table I. In general, based on the teachings herein, persons skilled in the art will recognize that the uncompressed, free-state (overall) diameter of RC-WVM implants 12 should not significantly exceed the largest anticipated fully extended IVC diameter for the patient in which the RC-WVM implant is to be used. RC-WVM implant height generally should be selected to balance implant stability at the monitoring position with geometry/flexibility/resilience providing the ability to fit in the intended region of the IVC without impacting either the hepatic or renal veins in the majority of the population, which could compromise sensing data produced by the implant. Height and stability considerations will be influenced, among other factors, by specific RC-WVM implant design configuration and whether or not distinct anchor features are included. Thus, as will be appreciated by persons skilled in the art, primary design considerations for RC-WVM implants 12 according to the present disclosure are provision of structures forming variable inductance L-C circuits with the ability to perform the measuring or monitoring function described herein, and which are configured to securely anchor the structures within the IVC without distortion of the IVC wall by providing adequate but relatively low radial force against the IVC wall.

TABLE I
RC-WVM Implant 12a & 12b
Example Dimensions

| Dim. | Element Name | Approximate Size (in millimeters) |
|---|---|---|
| A | Height | 10-100, typically about 20 |
| B | Strut length | 10-100, typically about 25 |
| C | Strut diam. | 0.1-2, typically about 1.5 |
| F | Anchor Length | 1-10, typically about 5 |
| | (extending) | |
| E | Anchor Length | 0.25-3, typically about 1.8 |
| | (barb) | |
| D | Overall | Three Sizes: |
| | Diameter | 20 mm/25 mm/32 mm +/− 3 mm |

Another alternative structure for RC-WVM implant 12 is illustrated by RC-WVM implant 12b as shown in FIG. 2A. Once again, the enlarged detail in the box of FIG. 2A represents a cross-sectional view taken as indicated. In this embodiment, implant 12b has an overall structure that is similar to that of implant 12a, formed on a frame with straight strut sections 38 and curved crown sections 40. In this embodiment, the discrete capacitor for the previous embodiment is replaced with distributed capacitance between the bundles of strands of wire. Multiple (for example, approximately fifteen) strands of wire 64 are laid parallel to each other and twisted into a bundle. This bundle is then wrapped, multiple times, around the entire circumference of wire frame 66 (which may be, for example, a 0.010″ diameter nitinol wire) resulting in multiple turns of parallel bundles of strands. The insulation between the bundles results in a distributed capacitance that causes the RC-WVM to resonate as previously. Overall dimensions are similar and may be approximated as shown in Table I. An outer, insulation layer or coating 60 may be applied either as previously described or using a dipping or spraying process In this case, the L-C circuit is created without a discrete capacitor, but instead by tuning the inherent capacitance of the structure through selection of materials and length/configuration of the wire strands. In this case, 20 turns of 15 strands of wire are used along with an outer insulation layer 60 of silicone to achieve a capacitance inherent in implant 12b in the range of approximately 40-50 ηF.

Unlike implant 12a, frame 66 of implant 12b is non-continuous so as to not complete an electrical loop within the implant as this would negatively impact the performance. Any overlapping ends of frame 66 are separated with an insulating material such as heat shrink tubing, an insulating epoxy or reflowed polymer. RC-WVM implant 12b (may or) may not include anchors. Instead, the implant is configured to have a compliance/resilience to permit it to move with changes in the IVC wall geometry or dimension while maintaining its position with minimal distortion of the natural movement of the IVC wall. This configuration can be achieved by appropriate selection of materials, surface features and dimensions. For example, the strut section length of the frame must balance considerations of electrical performance versus stability, wherein shorter strut section length may tend to improve electrical performance but longer strut section length may increase stability.

In order to energize RC-WVM implant 12 and receive the signal back from the implant, antenna module 16 will functionally include a transmit and a receive antenna (or multiple antennas). Antenna module 16 thus may be provided with physically distinct transmit and receive antennas, or, as in the presently described exemplary system 10, provided by a single antenna that is switched between transmit and receive modes. Antenna belt 16b, shown FIGS. 3 and 3A-D, illustrates an example of antenna module 16 employing a single, switched antenna. A single loop antenna is formed from a single wire and placed around the patient's abdomen. This wire antenna is connected directly to the control system 14.

In terms of mechanical construction, antenna belt 16b generally comprises stretchable web section 72 and buckle 74 with a connection for power and data link 24. In one embodiment, in order for the size of the antenna belt 16b to accommodate patients of different girths (e.g., a patient girth range of about 700-1200 cm), a multi-layer construction made up of a combination of high-stretch and low-stretch materials may be employed. In such an embodiment, base layer 76 is a combination of high-stretch sections 76a and low-stretch section 76b, which are joined such as by stitching. Outer layer 78, with substantially the same profile as base layer 76, may be comprised entirely of the high-stretch material, which may be a 3D mesh fabric. Within each section, antenna core wire 82 is provided in a serpentine configuration with an overall length sufficient to accommodate the total stretch of the section. Core wire 82 should not itself stretch. Thus, the stretchability of the fabric layers is paired with the core wire total length to meet the desired girth accommodation for a particular belt design. Outer layer 78 is joined along the edges to base layer 76. Stitching covered by binding material 80 is one suitable means for joining the two layers. The layers may be further bonded together by a heat fusible bonding material placed between the layers. End portions 81 of web section 72 are configured for attachment to buckle 74.

Core wire 82, which forms the antenna element, is disposed between the layers and provided with an extendable, serpentine configuration so that it may expand and contract with the stretch of the belt. A mid-section 84 of core wire 82, which corresponds to low-stretch section 76b, has a greater width. This section, intended to be placed in the middle of the patient's back with antenna belt 16b worn approximately at chest level at the bottom of the rib cage, provides greatest sensitivity for reading the signal from RC-WVM implant 12. As one possible example, core wire 82 may be made up of 300 strands of twisted 46 AWG copper wire with a total length in the range of approximately 0.5-3 m. For an antenna belt configured to stretch to accommodate patient girths in the range of about 700 to 1200 mm, the total length of core wire 82 may be approximately 2 m. In some embodiments, it may be preferable to place the antenna belt more caudally, with the height approximately at the height of the patient's elbows when standing.

Many ways of providing a workable buckle for such an antenna belt may be derived by persons of ordinary skill based on the teachings contained herein. Factors to be considered in designing such a buckle include physical security, ease of manipulation by persons with reduced dexterity and protection from electrical shock by inadvertent contact with the electrical connectors. As an example, buckle 74 is comprised of two buckle halves, inner half 74a and outer half 74b as shown in FIG. 3D. Buckle 74 provides not only physical connection for the belt ends, but also electrical connection for the antenna circuit formed by core wire 82. With respect to the physical connection, buckle 74 is relatively large in size to facilitate manipulation by persons with reduced dexterity. A magnetic latch may be employed to assist closure, for example magnetic pads 86a on inner buckle half 74a connect to magnetic pads 86b correspondingly disposed on buckle outer half 74b. If desired, the system can be configured to monitor for completion of the belt circuit and therefore detect belt closure. Upon confirmation of belt closure, the system may be configured to evaluate the signal strength received from the implant and an assessment made if the received signal is sufficient for a reading to be completed. If the signal is insufficient, an instruction may be provided to reposition the belt to a more optimal location on the patient.

Electrical connection of core wire 82 may be provided by recessed connector pins disposed on opposed connector halves 88a and 88b. Connection of power and data link 24 may be provided, for example, through a coaxial RF cable with coaxial connectors (e.g., SMA plugs) on buckle 74 and control system 14. As just one possible example, a convenient length for the power and data link, using a conventional, 50 Ohm coax cable, is about 3 m.

As mentioned above, use of a single coil antenna as in antenna belt 16b requires switching the antenna between transmit and receive modes. Such switching is executed within control system 14, an example of which is schematically depicted as control system 14a in FIG. 4. In this embodiment, control system 14a includes as functional modules 20 a signal generator module 20a and a receiver-amplifier module 20b. These functional modules, along with transmit/receive (T/R) switch 92 provide for the required switching of antenna belt 16b between the transmit and receive modes.

FIG. 3E schematically illustrates the interaction of the magnetic field [symbol] , created by antenna belt 16b, with RC-WVM implant 12. Both antenna belt 16b and implant 12 are generally disposed around an axis (A). For best results with a belt-type antenna, the axes around which each are disposed will lie in a substantially parallel orientation and, to the extent practicable, will lie coincident as shown in FIG. 3E. When properly oriented with respect to one another, current (I) in core wire 82 of antenna belt 16b generates magnetic field [symbol] , which excites the coil of implant 12 to cause it to resonate at its resonant frequency corresponding to its size/geometry at the time of excitation. An orientation between the antenna belt 16b and implant 12 as shown in FIG. 3E minimizes the power necessary to excite the implant coil and produce a readable resonant frequency response signal.

As with any RF coil antenna system, the antenna and system must be matched and tuned for optimum performance. Values for inductance, capacitance and resistance and their interrelationship should be carefully considered. For example, the coil inductance determines the tuning capacitance while the coil resistance (including the tuning capacitance) determines the matching capacitance and inductance. Given the relatively low power of the disclosed systems, special attention is given to these aspects to ensure that an adequately readable signal is generated by RC-WVM implant 12 upon actuation by the driving magnetic field. With an adjustable girth belt such as antenna belt 16b (or with different size antenna belts), additional considerations are presented because of the variable or different lengths of antenna coil controlled by the control system. To address these considerations, separate tuning-matching circuits 94, 96 (FIG. 4), as are understood in the art, are provided in signal generator module 20a and receiver-amplifier module 20b, respectively.

Using conventional coax cable for RF-power transmission, as is described above in one embodiment of power and data link 24, optimal RF power transfer between the antenna and the control system is achieved when the system and antenna impedances are matched to 50 Ohm real resistance. However, in the embodiment described above, resistance of antenna belt 16b is generally far below 50 Ohm. Transformation circuits, as part of tuning-matching circuits 94, 96, can be used to transform the antenna resistance to 50 Ohm. In the case of antenna belt 16b, it has been found a parallel capacitor transformation circuit is efficient for this purpose.

In one example of tuning using the system components heretofore described, a series capacitor was used, which, in conjunction with a matching capacitor, forms the total resonance. Using measured values as set forth below in Table II, a target resonance frequency was computed at 2.6 MHz based on the inductance and capacitance. Considering the inductance variation with stretching of antenna belt 16b at 2.6 MHz, the resonance frequency was measured to vary only from about 2.5 MHz to about 2.6 MHz for change in length between 1200 mm and 700 mm circumferences of antenna belt 16b, respectively. Considering the resistance of 11.1 Ohm, the Q-factor of the cable/belt assembly computes to be 3. Such a low Q-factor translates to a full width of the pulse at half maximum of 600 kHz. This is far less than the variation of the resonance frequency due to stretching of the belt from 700 mm to 1200 mm circumference. Tuning values for antenna belt 16b were thus determined at 2.6 MHz with C_{match}=2.2 nF and Cₜᵤₙₑ=2.2 nF.

TABLE II
Example of measured values for antenna belt 16b
Belt stretched to 28 cm dia. around water bottle

| | Resistance | Inductance |
|---|---|---|
| Point of measurement | [Ohm] | [10⁻⁶ H] |
| Measured at buckle | 0.3 | 1.69 |
| terminals with no cable | | |
| connected | | |
| Measured at output of | 11.1 | 3.03 |
| T/R switch 92 with 3 m | | |
| coax cable connected | | |

While it could be expected that a variable length antenna, such as included in antenna belt 16b might present difficulties in tuning and maintaining the antenna tuning as the length changed, it was discovered that with the present configuration this was not the case. As described above, by intentionally employing a cable for power and data link 24 that has a relatively large inductance compared to the antenna inductance, the proportional change in the inductance due to changes in belt diameter are small enough not to degrade performance.

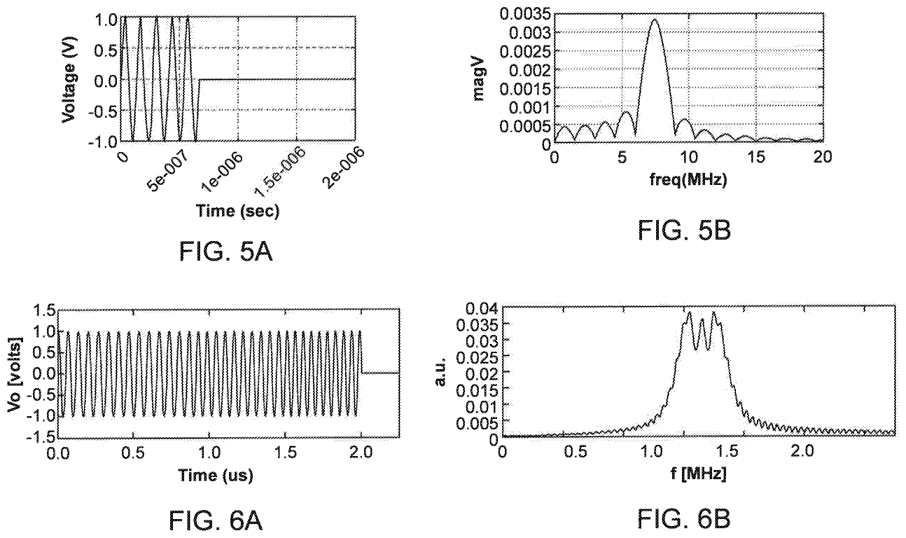

Referring again to FIG. 4, in addition to tuning-matching circuit 94, signal generator module 20a includes components that produce the signal needed for excitation of RC-WVM implant 12. These components include direct digital synthesizer (DDS) 98, anti-aliasing filter 100, preamplifier 102 and output amplifier 104. In one embodiment, the signal generator module 20a is configured to produce an RF burst excitation signal with a single, non-varying frequency tailored to a specific RC-WVM implant that is paired with the system (exemplary waveforms illustrated in FIGS. 5A and 5B). The RF burst comprises a predefined number of pulses of a sinusoidal waveform at the selected frequency with a set interval between bursts. The RF burst frequency value selected corresponds to the natural frequency of the paired RC-WVM implant 12 that would produce a lowest amplitude in the implant reader output. By doing this, optimum excitation is achieved for the worst case of implant response signal.

In an alternative implementation, control system 14 excites antenna module 16 at a predetermined frequency that is within an expected bandwidth of the paired RC-WVM implant 12. The system then detects the response from the paired RC-WVM implant and determines the implant natural frequency. Control system 14 then adjusts the excitation frequency to match the natural frequency of the paired implant and continues to excite at this frequency for a complete reading cycle. As will be appreciated by persons of ordinary skill, frequency determination and adjustment as described for this embodiment may be implemented via software using digital signal processing and analysis.

In another alternative implementation, each individual RF burst comprises a continuous frequency sweep over a predefined range of frequencies equal to the potential bandwidth of the implant (FIG. 6A). This creates a broadband pulse that can energize the implant at all possible natural frequencies (FIG. 6B). The excitation signal can continue in this “within burst frequency sweep mode” or the control system can determine the natural frequency of the sensor and adjust to transmit solely at the natural frequency.

In a further alternative implementation, the excitation comprises a transitory frequency sweep over a set of discrete frequency values covering the potential bandwidth of the paired RC-WVM implant 12. The frequency is sequentially incremented for each RF burst and the RMS value of the RC-WVM implant response is evaluated after each increment. Control system 14 then establishes the frequency that produces the maximum amplitude in RC-WVM implant response and continues exciting the paired RC-WVM implant at that frequency until a drop of a predefined magnitude is detected and the frequency sweep is re-started.

In yet another implementation, the excitation signal is composed of a pre-defined set of frequencies, wherein each remain constant. Control system 14 excites antenna module 16 (and hence the paired implant) by applying equal amplitude at all frequency components. The system detects the response from the paired implant and determines its natural frequency. Control system 14 then adjusts the relative amplitude of the excitation frequency set to maximize the amplitude of the excitation frequency that is closest to the natural frequency of the paired implant. The amplitude of the other frequencies are optimized to maximize the response of the paired implant while meeting the requirements of electro-magnetic emissions and transmission bandwidth limitations.

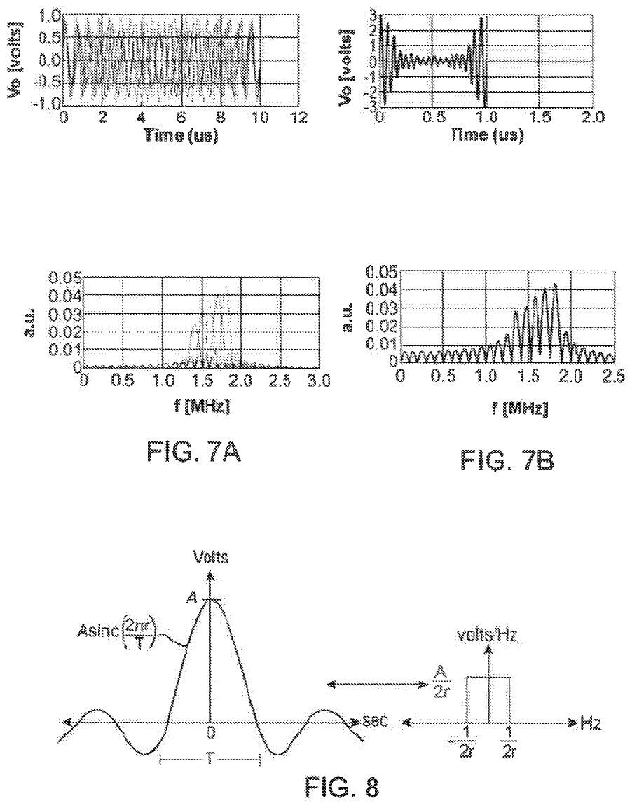

In another implementation, direct digital synthesizer (DDS) 98, may be provided as a multi-channel DDS system to generate a simultaneous pre-defined number of discrete frequencies belonging to the estimated operational bandwidth of the paired RC-WVM implant 12 as shown in FIGS. 7A and 7B. The magnitude of each frequency component thus may be independently controlled to provide the optimum excitation to a specific RC-WVM implant 12 based on its individual coil characteristics. Additionally, the relative amplitude of each frequency component can be independently controlled to provide optimum excitation to the implant, i.e., the amplitude of the frequency component is selected in such a way that in the worst case for the paired implant to transmit a response signal (i.e., most compressed) the excitation signal is maximized. In this arrangement all outputs from the multi-channel DDS system 98 are summed together using summing amplifier based on a high speed operational amplifier.

In yet another implementation, signal generator module 20a can be configured to provide pulse shaping as illustrated in FIG. 8. Arbitrary waveform generation based on direct digital synthesis 98 is employed to create a pulse of a predefined shape, the spectrum of which is optimized in order to maximize the response of the paired RC-WVM implant 12. The magnitude of the frequency components that result in decreased ring back signal amplitude is maximized while the magnitude of the frequency components that result in increased ring back signal amplitude is reduced, in order to obtain an approximately constant output signal amplitude and thus improved response from RC-WVM implant 12.

Referring again to FIG. 4, receiver-module 20b, in addition to tuning-matching circuit 96, includes components, e.g., single end input to differential output circuit (SE to DIFF) 106, variable gain amplifier (VGA) 108, filter amplifier 110 and output filters 112, for implant response detection, data conversion and acquisition for signal analysis. During the receive period, the T/R switch 92 connects the antenna belt 16b to the receiver-amplifier 20b, via the tuning and matching network 96. The response signal induced by the implant 12 in the antenna belt 16b is applied to a unity-gain single ended to differential amplifier 106. Converting from single-ended to differential mode contributes to eliminate common mode noise from the implant response signal. Since the amplitude of the implant response signal is in the microvolts range, the signal is fed, following conversion from single-ended to differential, into a variable gain differential amplifier 108 that is able to provide up to 80 dB (10000 times) voltage gain. The amplified signal is then applied to a active band-pass filter-amplifier 110 to eliminate out-of-band frequency components and provide an additional level of amplification. The resulting signal is applied to passive, high-order low pass filters 112 for further elimination of out-of-band high frequency components. The output of the filter is fed into the data conversion and communications module 22. The data conversion and communications module 22 includes components to provide data acquisition and transfer from the electronic system to the external processing unit. A high-speed analog-to-digital converter (ADC) 114 converts the output signal of the receiver module 20b into a digital signal of a predefined number of bits (e.g., 12 bits). This digital signal is transferred in parallel mode to microcontroller 116. In one implementation, a level shifter circuit is used to match the logic levels of the ADC to the microcontroller. The data outputted by the ADC is sequentially stored in internal flash memory of the microcontroller. To maximize the data throughput, direct memory access (DMA) is used in this process. Microcontroller 116 is synced with the direct digital synthesizer 98, so data acquisition starts when an RF burst is transmitted for excitation of implant 12. Once triggered, the microcontroller captures a predefined number of samples (e.g. 1024). The number of samples multiplied by the sampling period defines the observation window over which the response signal from implant 12 is assessed. This observation window is matched to the length of the response signal from implant 12, which depends on the time constant of the signal decay.

As a means of noise reduction, the response signal of the implant 12 is observed a predefined number of times (e.g., 256), and the average response is then computed. This approach greatly contributes to increasing the signal-to-noise ratio of the detected signal.

The average response is then transmitted to an external interface device 18 (e.g., laptop computer) by means of communications module 118. Different approaches can be taken for this. In one embodiment, the communication is performed using the UART interface from the microcontroller and external hardware is employed to convert from UART to USB. In a second embodiment, a microcontroller with USB driving capabilities is employed, and in this case connection with the external interface device is achieved by simply using a USB cable. In yet another implementation, the communication between the microcontroller and the external interface device is wireless (e.g. via Bluetooth).

The system is to be powered by a low voltage power supply unit (PSU), consisting of a AC-DC converter with insulation between mains input and output providing a minimum of 2 Means of Patient Protection (MOPP) as per Clause 8 of IEC 60601-1:2005+AMD1:2012. In this way, the power supply provides protection from electrocution to the user. The PSU is able to accommodate a wide range of mains voltages (e.g., from 90 to 264 VAC) and mains frequencies (e.g., 47 to 63 Hz) to allow operation of the system in different countries with different mains specifications.

Control system 14a as described above utilizes a software-based frequency detection. Thus, in terms of signal transmission, once the excitation frequency is optimized, system 10 employing control system 14a with signal generator module 20a operates in open loop mode, i.e., frequency or frequencies and amplitude of the transmit signal are not affected by RC-WVM implant 12 response. On the receive side, using amplifier-receiver module 20b, control system 14a detects the response signal from RC-WVM implant 12 and such signal is digitized using a high-speed data converter. The raw digitized data is subsequently transferred to a processing unit (e.g., laptop computer or other equipment microcontroller) and digital signal analysis techniques (e.g. Fast Fourier Transform) are applied to establish the frequency content of the signal. Thus, one advantage of using these software-based techniques is that phased-lock loop (PLL) circuits or similar circuits are not used or required in control system 14a.

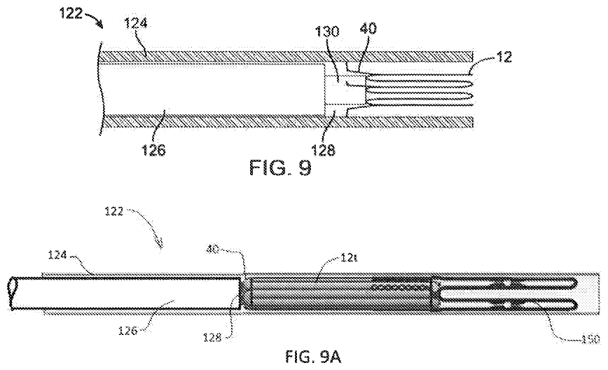

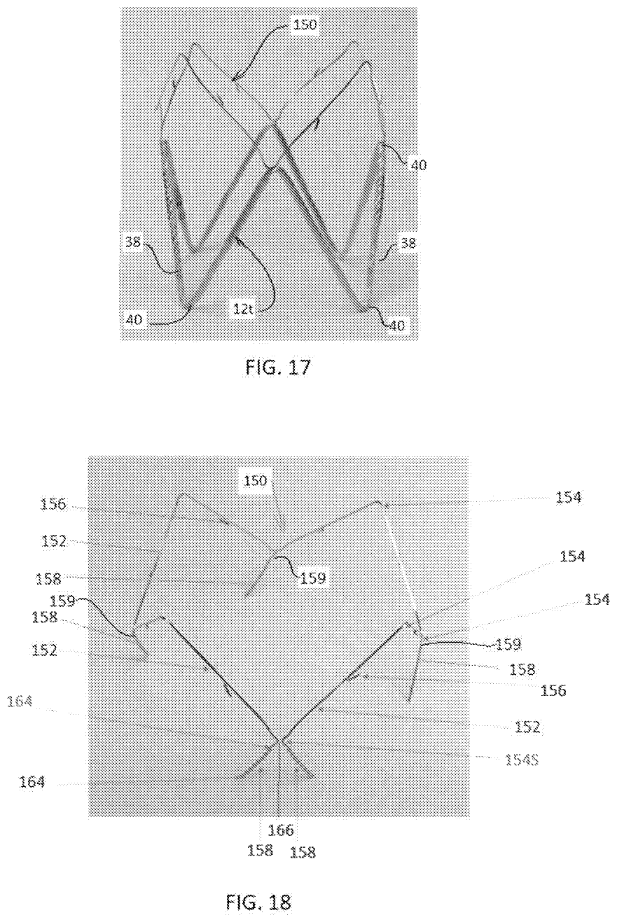

A further component of the overall RC-WVM system as described herein is the RC-WVM implant delivery system. FIGS. 9 and 9A schematically illustrate aspects of intravascular delivery systems for placing RC-WVM implants 12 at a desired monitoring location within the IVC, which may generally comprise delivery catheter 122 including outer sheath 124 and pusher 126 configured to be received in the lumen of outer sheath 124. In general, insertion of devices into the circulatory system of a human or other animal is well known in the art and so is not described in detail herein. Those of ordinary skill in the art will understand after reading this disclosure in its entirety that RC-WVM implants 12 can be delivered to a desired location in the circulatory system using, e.g., a loading tool to load a sterile RC-WVM implant into a sterile delivery system, which may be used to deliver an RC-WVM implant to the IVC via a femoral vein or other peripheral vascular access point, although other methods may be used. Typically RC-WVM implant 12 will be implanted using a delivery catheter, delivery catheter 122 being an illustrative example thereof, and the RC-WVM implant will be optimized for delivery through as small a catheter as possible. To facilitate this, bends at the implant crown sections 40 may be small-radius bends to facilitate a low profile when packed into the delivery catheter as shown. In one alternative, pusher 126 may be provided with a stepped distal end 128 having a reduced diameter end portion 130 configured to engage the inner perimeter of RC-WVM implant 12 when compressed for delivery. For implant embodiments employing anchors such as anchors 48 in FIG. 2 or anchors 48s in FIG. 11A et seq. end portion 130 may be configured to engage an inner perimeter defined by the anchors in the compressed configuration as illustrated in FIG. 9. Alternatively, pusher distal end 128 may be provided with a straight, flat end or other end shape configured to cooperate with a specific RC-WVM implant and anchor design. For example, as shown in FIG. 9A, RC-WVM implant 12t with anchor frame 150 (see, e.g., FIGS. 17 and 18) may be deployed with a flat distal end pusher 128, which bears against crown sections 40 of implant 12t, with anchor frame 150 disposed opposite pusher 128.

In one deployment option, an RC-WVM implant may be inserted from a peripheral vein such as the femoral or iliac vein into the IVC to be positioned at a monitoring location between the hepatic and renal veins. It will be understood that the implant also may be introduced from other venous locations. Depending on implant configuration, when placed in the IVC for fluid status monitoring, specific orientation of RC-WVM implant 12 may be required to optimize communication with the belt reader antenna coil. To facilitate desired placement or positioning, the length and diameter of RC-WVM implant 12 may be designed so that it gradually expands (“flowers”) as it is held in position with the pusher 126 and the sheath 124 is withdrawn. Such a gradual, partial deployment helps ensure that RC-WVM implant 12 is properly positioned in the IVC. The sensor length to vessel diameter ratio (where the length is always greater than the vessel diameter) is also an important design factor to ensure that the sensor deploys in the correct orientation in the IVC. In a further alternative, distal end 128 of pusher 126 may be configured to releasably retain the anchors or a proximally oriented portion of the implant before it is fully deployed from outer sheath 124 so that it may be retracted for repositioning as needed. For example, small, radially extending studs may be provided near the end of end portion 130, which engage behind the proximal crowns of implant 12 so long as it is compressed within outer sheath 124 whereby the implant may be pulled back in from a partially deployed position, but self-releases from the studs by expansion when fully deployed after positioning is confirmed. Conventional radiopaque markers may be provided at or near the distal ends of outer sheath 124 and/or pusher 126, as well as on RC-WVM implant 12 to facilitate visualization during positioning and deployment of the implant. Typically, where anchor features are employed, the implant will be positioned with the anchor features proximally oriented so the anchors are the last portion deployed in order to facilitate correct orientation within the IVC and potentially allow for pull back and repositioning as may be needed. Once the implant is fully deployed, delivery catheter 122 may be withdrawn from the patient, leaving implant 12 as a discrete, self-contained unit in the vessel without attached wires, leads, or other structures extending away from the monitoring location.

### Example 1

Systems as described herein have been evaluated in pre-clinical testing using RC-WVM implant 12a (as in FIG. 2), an antenna belt similar to antenna belt 16b (as in FIG. 3) and control system 14a (as in FIG. 4). The implants were deployed into ovine IVCs using delivery systems 122 (as in FIG. 9) using standard interventional techniques. Deployment was confirmed angiographically, using intravascular ultrasound and using the antenna belt.

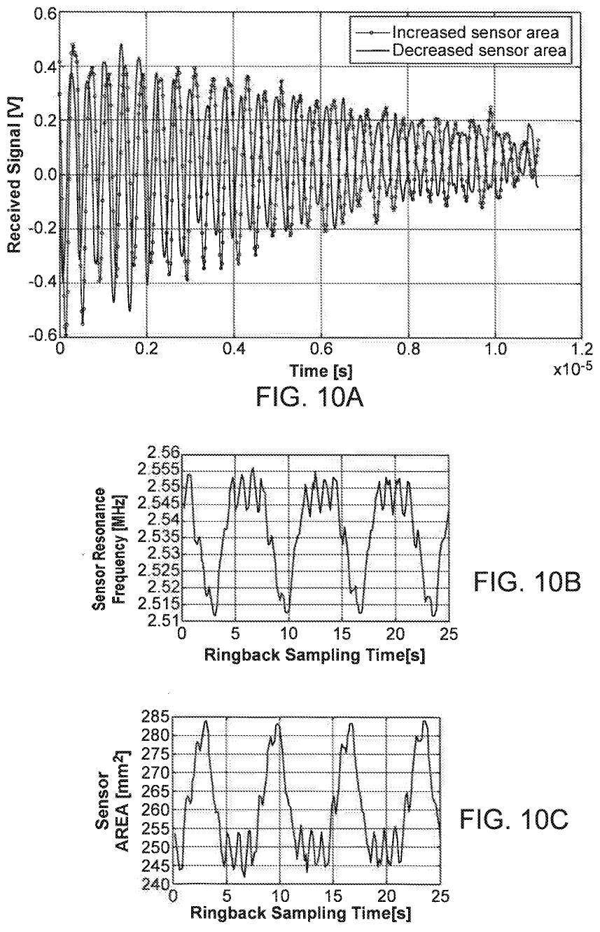

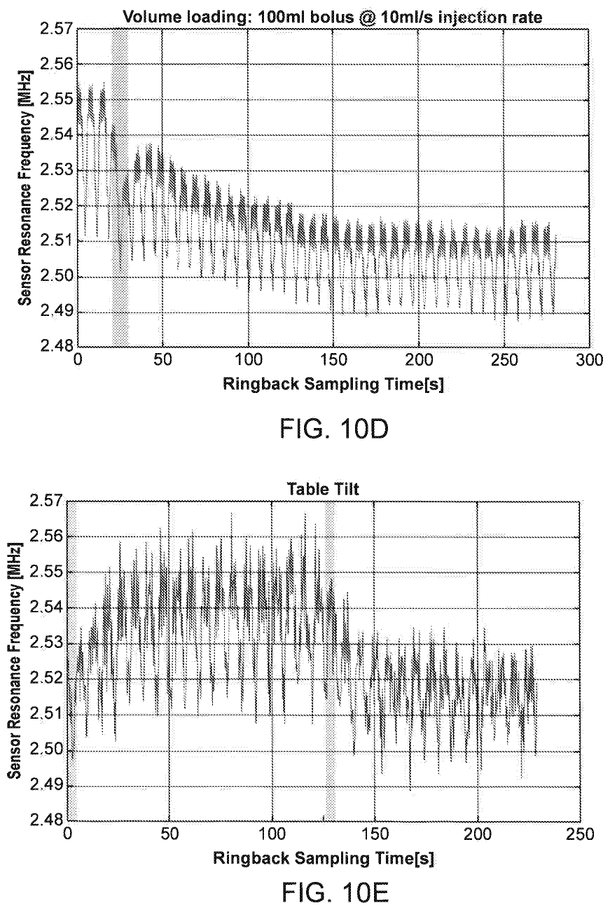

FIGS. 10A, 10B and 10C illustrate, respectively, the raw ring down signal, detection of the maximum frequency and conversion of this to an IVC area using a reference characterization curve. FIG. 10A shows the raw ring down signal in the time domain with the resonant response of the RC-WVM implant decaying over time. Modulation of the implant geometry results in a change in the resonant frequency which can be seen as the difference between the two different plotted traces. FIG. 10B shows the RC-WVM implant signal as converted into the frequency domain and plotted over time. The maximum frequency from FIG. 10A is determined (e.g., using fast Fourier transform) and plotted over time. The larger, slower modulation of the signal (i.e., the three broad peaks) indicate the respiration-induced motion of the IVC wall, while the faster, smaller modulation overlaid on this signal indicate motion of the IVC wall in response to the cardiac cycle. FIG. 10C shows the frequency modulation plotted in FIG. 10A converted to an IVC area versus time plot. (Conversion in this case was based on a characterization curve, which is determined through bench testing on a range of sample diameter lumens following standard lab/testing procedures.) FIG. 10C thus shows variations in IVC area at the monitoring location in response to the respiration and cardiac cycles.

The ability of RC-WVM implant 12 (in this case, implant 12a) to detect IVC area changes as a result of fluid loading is demonstrated in FIGS. 10D and 10E. In one example, the results of which are shown in FIG. 10D, after placement of RC-WVM implant 12 in the ovine IVC and confirmation of receipt of the implant signal, a fluid bolus of 100 ml at 10 ml/s was added to the animal. The grey band in FIG. 10D indicates the administration of the fluid bolus. As reflected by the decreasing frequency ring-back signal from RC-WVM implant 12, the added fluid volume caused the IVC to expand, and with it the implant, which in turn causes a change in the inductance of the implant thus changing the frequency of its ring-back response to excitation. In another example, with results shown in FIG. 10E, the operating table was tilted to shift fluid within the animal. Starting from the left in FIG. 10E, the first grey band indicates the time when the table was initially tilted. Tilting of the table caused fluid to shift away from the IVC, causing the IVC to reduce in diameter, and thus increasing the frequency of the ring-back signal of RC-WVM implant 12 as it moved to a smaller diameter with the IVC. The second grey band indicates the time when the table was returned from tilted to flat. At this point, fluid shifts back into the IVC, causing it to increase in size with the added fluid volume and thus reduce the frequency of the ring-back signal as explained above.

These output signals thus demonstrate the detection of modulation of the IVC with respiration. In particular, it will be appreciated that embodiments of the present invention can thus provide an unexpectedly powerful diagnostic tool, not only capable of identifying gross trends in IVC geometry variations, but also capable of discriminating in real-time between changes in IVC geometry arising from respiration and cardiac function.

### RC-WVM Implant Design Considerations and Alternative Implant Embodiments

It will be appreciated that the measurement of dimensional changes in the IVC presents unique considerations and requirements arising from the unique anatomy of the IVC. For example, the IVC is a relatively low pressure, thin-walled vessel, which changes not simply its diameter, but its overall shape (cross-sectional profile) in correspondence to blood volume and pressure changes. Rather than dilating and constricting symmetrically around its circumference, the IVC expands and collapses primarily in the anterior-posterior direction, going from a relatively circular cross-section at higher volumes to a flattened oval-shaped cross-section at lower volumes. Thus embodiments of RC-WVM implants 12 must monitor this asymmetrical, low-pressure collapse and expansion in the A-P direction without excessive radial constraint, yet must also engage the vessel walls with sufficient force to anchor the implant securely and prevent migration. Accordingly, RC-WVM implant 12 must be capable of collapsing with the vessel in the A-P direction from a generally circular cross-section to an oval or flattened cross-section without excessive distortion of the vessel's natural shape. These requirements are achieved according to various embodiments described herein by appropriate selection of material compliance and configuration such that the coil measurement section of RC-WVM implant 12 is maintained in contact against the IVC wall without undue radial pressure that may cause distortion thereof. For example, RC-WVM implants 12 according to embodiments described herein may exert a radial force in the range of about 0.05 N-0.3 N at 50% compression. In another alternative, potentially increased security of positioning may be achieved without compromising measurement response by physically separating anchoring and measurement sections so as to move possible distortions of the vessel wall due to anchoring a sufficient distance spaced from the measurement section so as not to affect measurements.

RC-WVM implants 12 as described may be configured in various structures such as collapsible loops or tubes of formed wire with resilient sinusoidal or “Z-shaped” bends, or as more complex collapsible shapes with more resilient regions such as “spines” joined by relatively less resilient regions such as “ears.” Each structure is configured based on size, shape and materials to maintain its position and orientation through biasing between resilient elements of the implant to ensure contact with the vessel walls. Additionally, or alternatively, anchors, surface textures, barbs, scales, pin-like spikes or other securement means may be placed on the structure to more securely engage the vessel wall. Coatings or coverings also may be used to encourage tissue in-growth. In some embodiments it may be preferable to configure specific portions of the structure, for example the coil spines, as the position-maintaining engagement portion in order to reduce any effect of the biasing force on movement of the vessel walls as sensed at the coil ears, or vice-versa. In yet other embodiments, separate anchoring structures may be coupled to a coil-measurement portion of the implant. Such anchoring structures may comprise hooks, expandable tubular elements, or other tissue-engaging elements which engage the vessel upstream or downstream of the coil portion so as to minimize any interference with the natural expansion or contraction of the vessel in the area of the coil itself. Sensing modalities and positioning is described in more detail below.

When RC-WVM implant 12 is energized it must generate a signal of sufficient strength to be received wirelessly by an external system. In the case of a variable induction circuit, the coil which transmits the signal to the external receiver must maintain a tubular shape or central antenna orifice of sufficient size, even when the vessel is collapsed, such that its inductance is sufficient to generate a field strong enough to be detected by an external antenna. Thus, in some embodiments, it may be desirable that the variable inductor have a collapsing portion which deforms with the expansion and collapse of the vessel, and a non-collapsing portion which deforms relatively little as the vessel collapses and expands. In this way, a substantial portion of the coil remains open even when the vessel is collapsed. In other embodiments, the coil may be configured to deform in a first plane containing the anterior-posterior axis while deflecting relatively little in a second orthogonal plane containing the medial-lateral axis. In still other embodiments, a first inductive coil may be provided to expand and collapse with the vessel, and a separate transmit coil, which deforms substantially less, provided to transmit the signal to the external receiver. In some cases, the transmit coil also may be used as an anchoring portion of the implant.

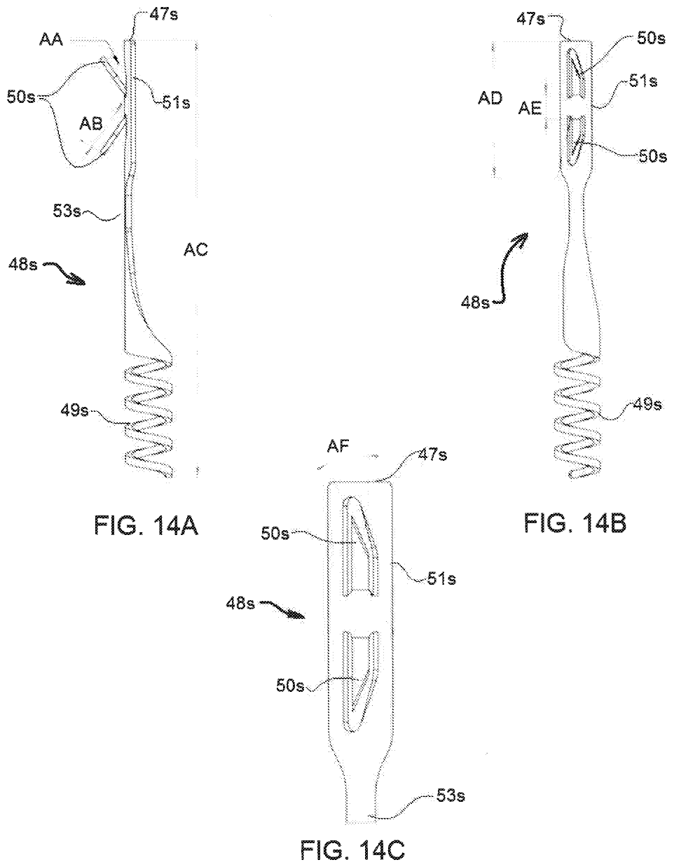

Turning to specific alternative RC-WVM implant embodiments disclosed herein, a first exemplary alternative embodiment is RC-WVM implant 12s, shown in FIGS. 11A, 11B, 11C, and alternative anchor 48s shown in FIGS. 14A, 14B and 14C.

RC-WVM implant 12s utilizes PTFE coated gold Litz wire 42s wound on nitinol wire frame 44s. PTFE has good heat resistance to withstand manufacturing processes while also being biocompatible. The overall configuration of implant 12s includes strut sections 38 and crown sections 40 substantially as described above. Alternatively, anchors 48s are secured adjacent crown sections 40 as described below. Sections of heat shrink tubing 61s are used to help ensure compression of reflow material and may be removed in a later assembly step. A section of heat-shrink tubing 60s may be used to cover and insulate capacitor 46s, which in one embodiment may be a 47 nF capacitor, or heat shrink tubing also may be removed as mentioned above.

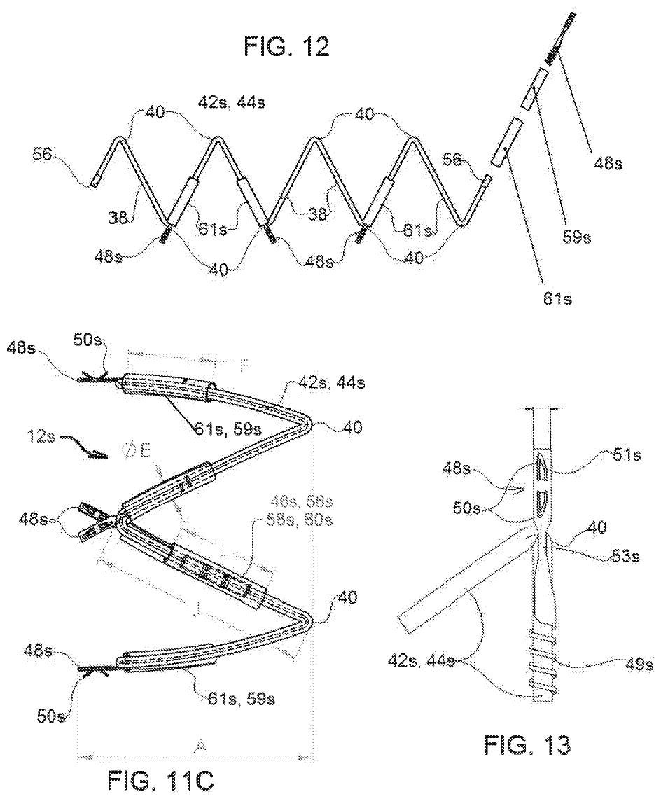

Capacitor 46s may be comprised of any suitable structure to provide the desired capacitance, in one embodiment 47 nF, as mentioned. For example, the desired capacitance may be achieved with a specifically sized gap, different terminal materials (e.g., leads, etc.), overlapping wires, or it could be a gap in a tube with a certain dielectric value. In an exemplary embodiment as illustrated, surface mount capacitor 46s is soldered between the two terminals 56s, formed through the joining of the 300 strands of Litz wire 42s. Other electrical attachments such as crimped, or attached directly to the terminals of the cap brazed with no solder may also be employed. The capacitor section is then encapsulated using a reflow process comprising positioning polymer reflow tube 59s over the capacitor, connection and terminals, followed by heat-shrink tubing 60s positioned over the reflow tube. Reflow tube 59s and heat shrink tube 60s are placed over the Litz wire/nitinol frame assembly before the capacitor before the capacitor is soldered in place (FIG. 12 illustrates the reflow and heat shrink tubes for the anchor, which are similarly positioned). The tolerances on the O.D.s of these tubes and their fit is selected to facilitate assembly, minimize overall profile of the final implant configuration, and optimize the flow of the material to increase bond strength. Heat is then applied to melt the polymer tube and shrink the heat-shrink, thus compressing the molten polymer over the capacitor forming a seal. The heat shrink tube is then removed. Alternative designs may employ over-moulding processes, a dipping process, epoxy potting or similar processes using appropriate biocompatible materials.

Details of alternative anchors 48s are shown in FIGS. 14A-C. Anchors 48s are generally formed with at least two sections, an attachment section 49s where the anchor is fixed to the implant and an anchor section 51s, which provides fixation to the vessel wall. In some embodiments, as shown in FIGS. 14A-C, an additional isolation section 53s is interposed between the anchor and attachment sections to allow independent mechanical motion between the anchor section and the attachment section in order to help isolate effects of the anchors acting on the vessel wall from the sensing function of the implant. Multiple anchors 48s may be used for an anchor system, wherein plural attachment sections 49s form an anchor system attachment section and plural anchors or anchor sections 48s form an anchor system anchor section.

Anchor 48s may be formed by laser cutting a pattern from a nitinol tube and shape setting the anchor barbs via a heat treatment process. Other embodiments can be formed using wire of various materials, shape set or bent using a standard process, or laser cut from other metals or bioabsorbable polymers. External surfaces of anchors may utilize different shapes of anchors or different surface finishes to engage the vessel wall and prevent migration of the implant. The overall length of anchors 48s that extend beyond crowns sections 40 of implant 12s is selected to facilitate the expansion of the implant upon deployment from delivery system 122 (FIG. 9) while minimizing the impact of the movement of the implant with the motion of the vessel. This occurs as described above when the distal end of the implant is partially ejected out of outer sheath 124 and engages with the vessel wall. Length of anchor protrusion is selected to allow the expansion to effectively occur. If the protrusion is too long, the implant may not deploy in an expanding, flowering manner as desired. In one embodiment the protrusion of the anchor beyond crown sections 40 (dimension D in FIG. 11B) is less than the inner diameter of outer sheath 124 of the delivery catheter.

Attachment section 49s may be formed using a tube laser cutting process to produce a spiral section of a tube. As indicated in FIG. 12, each anchor 48s is positioned by winding the spiral of the attachment section around the sensor strut. In one embodiment, the internal dimension of the spiral portion of the attachment section is less than the outer dimension of the implant strut 38 so that an interference fit is formed, thus securing the anchor in position. In another embodiment, the internal dimension of the spiral portion is less than the outer dimension of terminal 56s, but greater than the outer dimension of implant strut 38 and can therefore be moved once wrapped into position on the strut. In one illustrative example, with an implant coil strut having a nominal diameter of approximately 1.143 mm, the inner diameter of the attachment section spiral may be about 1.156±0.05 mm (with an outer diameter of about 1.556±0.05 mm). In general, relative dimensions of the implant coil O.D. and anchor spiral I.D. may be selected so as to provide a locational interference fit.

After placement of the anchor on the implant strut, polymeric reflow tube 59s is positioned over this assembly and further heat shrink tube 61s placed over this. Heat is then applied to melt the polymer tube and shrink the heat shrink tube, thus forcing the polymer between spacing in the spiral of the anchor section and thereby reinforcing the fixation of the anchor to the implant assembly. Reflow tube 59s also may be sized with a slight interference fit between the outer surface of the implant assembly and the inner surface of the anchor attachment section to provide some fixation, both longitudinal and rotational, during assembly. The spacing between the spirals is designed to allow the reflow material to flow into the spaces and form a bond. The width of the spirals is designed to allow the spiral section to be manipulated into position during assembly, while still providing sufficient rigidity when fully assembled. The thickness of the section is minimized to reduce the overall profile of the implant. One advantage of attachment section 49s employing a spiral portion as means of attachment is that it permits attachment of the anchor to any wire-based implant, including insulated wire implants without disturbing or penetrating the insulation layer. The spiral portion as described distributes the attachment force across space of the insulation layer to avoid compromise of the layer and the spaces between the spiral facilitate bonding attachment. Another advantage of attachment using a spiral portion as described is that the aspect ratio of the spiral section may be selected so as to allow the spiral to be slightly unwound to permit placement of the anchor in the middle of the implant strut section without needing to thread it over the end past the capacitor terminals. Alternative embodiments of attachment section 49s may employ other shapes, such as a T-shape rather than the spiral section, to prevent rotation and detachment from the sensor. Further alternatives may also include the replacement of polymer reflow tube 59s with just heat-shrink that could be left in place, or use an adhesive or other bonding technology.

As shown in FIGS. 14A-C, anchor section 51s comprises two, laser cut and shape set anchor barbs 50s. The barbs 50s are positioned on the vessel facing surface of the anchor and are angled in some embodiments at between about 10 and 80 degrees to provide fixation with the vessel wall, resistance to cranial and caudal implant migration and to also facilitate collapse for loading and deployment of the implant through its delivery system. Barbs 50s are shaped to point to engage with the vessel wall and have a length sufficient to penetrate into the vessel without perforating through it, typically between about 0.5 and 2.0 mm. The distal end of anchor section 51s may have a flat end surface 47s to engage with the pusher of the deployment system and may be filleted to avoid any sharp edges that may cause unnecessary vascular response or catch on the delivery system. Other alternative embodiments may include multiple barbs or different surface treatments or barb shapes to optimize vessel fixation.

Isolation section 53s is designed to isolate or reduce transmission of mechanical motion of anchor section 51s from or to attachment section 49s and thus to the implant, to allow the implant to move freely and at least substantially free of distortions resulting from contact of the anchor section with the vessel wall. Isolation section 53s thus may comprise a narrow cross-section area to provide flexibility while keeping thickness constant to provide adequate support. Fillets/curves surfaces as shown are maintained to avoid stress concentrations that could lead to fatigue or unwanted tissue damage. Alternative embodiments of isolation section 53s may include varying tube thickness to provide more flexibility or varying the cross-section in a non-mirrored fashion to provide preferential flexibility in one direction.

FIGS. 15A and 15B show alternative embodiments for antenna belt module 16s. In order to accommodate patients of different girth, belt antenna 16s employs a loop antenna wire 82s mounted on or within base layer 76s, which is wrapped around the patient to form a non-continuous circumferential loop. Communications link 24s is provided substantially as described above. By using a loop core wire, the core wire forms a loop antenna without having to extend all the way around the patient. In this manner, the buckle or clasp (not shown) that closes the belt need not also provide electrical connections to complete the antenna loop. A simplified clasp may therefore use a variable connection method such as Velcro or other connection means, thus removing the need for multiple size belts. As shown in FIGS. 15A and 15B, antenna belt 16s utilizes a single (or multiple) loop core wire 82s wrapped around the patient. Loop ends 83s of core wire 82s should be substantially adjacent when the base layer is wrapped around the patient, typically within about 2 cm to about 10 cm apart. Depending on specific design parameters the signal strength provided by discontinuous looped core wire 82s may be less than provided by continuous circumferential core wire 82 as described above. However, depending on application and specific clinical requirements, the simplified clasp and ease of use offered by antenna belt module 16s may offer usability advantages that outweigh the signal requirements.

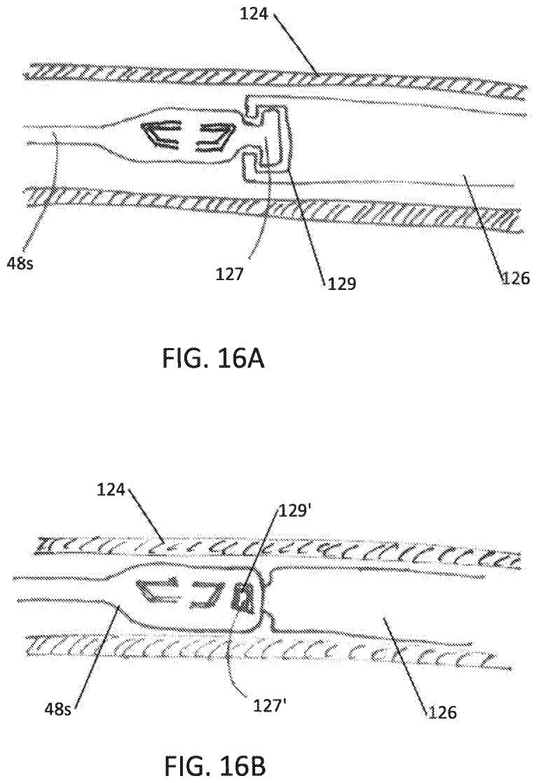

Implant repositionability or even recapture with the deployment system can be facilitated through the addition of recapture features in the distal end of the anchor and the pusher tip, exemplary embodiments of which are shown in FIGS. 16A and 16B. Such recapture features allow the sensor to remain attached to the pusher while being partially deployed. From this point the sensor can be fully deployed using the mechanism, the device repositioned as the sensor is still attached to the pusher, or recaptured by advancing the sheath over the sensor and the removal of the sensor. These features can take many forms including interlocking elements, screws, or release bumps. In one embodiment, as illustrated in FIG. 16A, recapture features 127, 129 may include a “T shaped” extension 127 to the anchor, which engages with an appropriately shaped recess 129 in the distal end of pusher 126. In another alternative, shown in FIG. 16B, recapture features 127′, 129′ include through-hole 127′ in the distal end of the anchor through which pin-shaped extension 129′ from pusher 126 engages to provide engagement while retained within outer sheath 124. Such recapture features could be used to partially deploy the sensor, while retaining the ability to reposition or recapture it. The recapture features remain engaged while the distal end of the anchor remains within the sheath. When the operator is satisfied with the final position, the sheath would be withdrawn fully, thus releasing the interlocking features and deploying the sensor.

While anchors 48s are shown in FIGS. 11A-C as attached only at one end of the implant (to facilitate flowering deployment as described), it is contemplated that anchors may be placed at both ends of an implant, with fewer or more anchors provided as compared to the four shown in the figures.

In other alternative embodiments, as illustrated in FIGS. 17-29D, one or more anchor elements to help prevent migration may be provided as an integrated anchor frame, as opposed to individual anchor elements described hereinabove. In one example, as shown in FIG. 17, an RC-WVM implant comprises anchor frame 150 is attached to RC-WVM sensor section 12t. The RC-WVM sensor section (or just “sensor section”) 12t may comprise any previously described “Z-shaped” coils or similar RC-WVM implant 12 as described above generally comprising strut sections 38 joined by crown sections 40. For the sack of clarity, hereinafter, with respect to embodiments described in reference to FIGS. 17-29D, RC-WVM implant (or “implant” alone) refers to the combined RC-WVM sensor section and anchor frame 150. Anchor frame 150 may be formed of nitinol wire or laser cut tubing whereby the tube is expanded to the equivalent diameter of the sensor section. Nitinol, or other materials with similar properties, is well-suited as material for anchor frame 150 because it allows the anchor frame to collapse to the same loaded configuration in the loader as the RC-WVM sensor section (see FIG. 9A.)

FIG. 18 shows an example of anchor frame 150 before it is attached to a sensor section, such as RC-WVM sensor section 12t. Similar to RC-WVM sensor section 12t, anchor frame 150 comprises a series of straight strut sections 152 (also referred to as anchor sections) joined by curved crown sections 154 to forma resilient, concentric zig-zag or linked “Z-shapes” structure, which may also be considered to be sinusoidal in appearance. One or more anchor barbs 156 are disposed within the strut sections or anchor sections as described in more detail below. Anchor frame 150 as shown in FIGS. 17 and 18 includes only a single anchor barb 156 on each strut section 152. Anchor frame 150 is attached to the sensor section by attachment arms 158 that overlap strut sections 152 of the sensor section. Note also that crown sections 154 on the end opposite attachment sections may be provided with recapture features such as recapture features 127, 127′, as shown in FIGS. 16A and 16B, which mate with corresponding recapture features 129, 129′ formed on the distal end of deployment pusher 126.

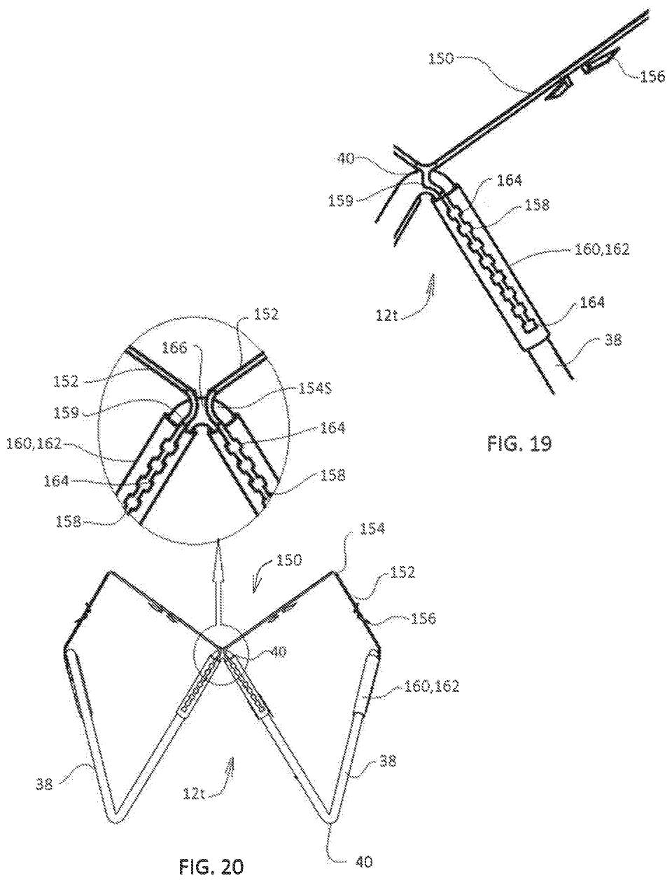

As best seen in detail in FIG. 19, polymeric reflow tube 160 is positioned over attachment arm 158 and further heat shrink tube 162 placed over the reflow tube. As illustrated in FIG. 19, attachment arm 158 is visible through transparent reflow and heat shrink tubes 160 and 162. Heat is then applied to melt polymer reflow tube 160 and shrink the heat shrink tube 162, thus forcing the polymer between and around attachment arm 158 and thereby fixing anchor frame 150 to the RC-WVM sensor section. Reflow tube 160 may be sized with a slight interference fit between the outer surface of strut section 38 and an inner surface of the reflow tube to provide some stability, both longitudinal and rotational, during assembly. Attachment arms 158 may be configured to include an anchor isolation section 159. Isolation section 159 is one form isolation means as previously described. Radial force requirements of anchor frame 150 and the function of isolation section 159 are also discussed in more detail below.

Attachment arm 158 may contain a saw tooth-like configuration as shown in FIG. 19 wherein spaces between teeth 164 allow the reflow material to flow in between and form a more secure bond. Other, alternative configurations for attachment arms 158, which provide this increased surface are considered to increase the bond strength such as zig zags, T-connectors, S connectors, and voids in center of struts are shown, respectively, in FIGS. 29A-D. Further alternatives include surface finishes or texturing on attachment arms 158. In certain designs such alternative configurations may permit the thickness of the attachment arm to be minimized to reduce the overall profile of the implant.

In some embodiments, for example as shown in FIGS. 18 and 20, it may be desirable to provide split 166 in anchor frame 150 so as to not produce a continuous ring of conductive material that could cause interference with sensor readings. Split 166 provides a break in the anchor frame to prevent the magnetic field from the external reader coupling into the anchor frame and potentially providing interference from the RC-WVM implant signal generated by the sensor section. Split 166 in anchor frame 150 advantageously is located at close to the sensor section, for example approximately at the center of an anchor frame crown 154 so that the split in the frame does not significantly compromise structural integrity of anchor frame 150. In one such example, as shown in FIGS. 18 and 20, split crown 154S is provided with double attachment arms 158, one securable to each strut section 38 on opposite sides of corresponding implant crown section 154. In other embodiments, the split may be located elsewhere on the anchor frame as further described below. If desired, double attachment arms 158 may be provided for non-split anchor crowns 154 as well.

In other embodiments, the decoupling split 166 of the anchor frame may be located elsewhere on the frame and, in such cases, preferably structurally reinforced by bridging with an additional metallic or polymeric component that provides sufficient structural integrity to the anchor frame while maintaining the discontinuous configuration. Alternatively, a continuous anchor frame structure may be devised by carefully selecting the amount of metallic material of the frame and shape of the frame to minimize or control interference with the RC-WVM implant signal such that it may be otherwise compensated for in signal processing.

In some embodiments, anchor frame 150 may be attached to the RC-WVM sensor section and loaded in the deployment system with the orientation of the anchor frame exposed first during deployment. In this case, pusher 126 of delivery system 122 bears on crown sections 40 of the sensor section (see, e.g., FIG. 9A). In other embodiments this configuration may be reversed, with the sensor section deployed first and the pusher of the deployment system bearing on crowns 154 of anchor frame 150. The orientation may be varied depending on factors such as the access site for implantation, e.g. femoral vein versus jugular vein. In a further alternative, as shown in FIG. 21, for increased anchoring an anchor frame 150 may be provided on each end of the RC-WVM implant (such as sensor section 12t), in which case the anchor frame would be first deployed regardless of orientation of the RC-WVM implant in the delivery system.

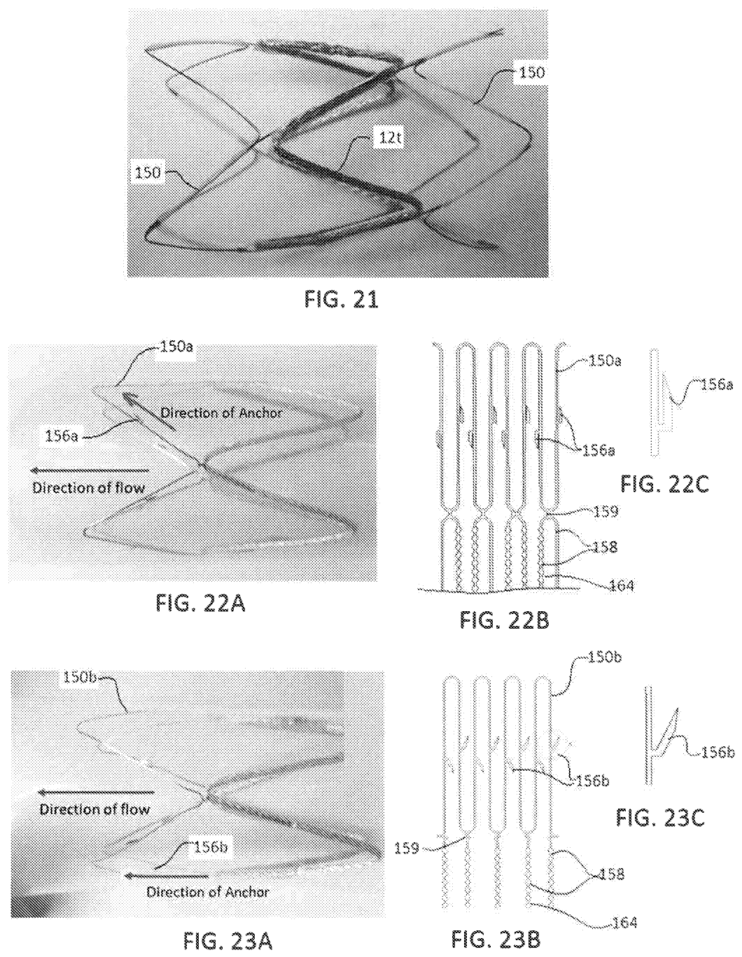

Once an RC-WVM implant employing anchor frame 150 is deployed within a vessel, barbs 156 engage with the vessel wall in various orientations to prevent movement of the device. FIGS. 22A, 22B and 22C show one embodiment of anchor frame 150a in which anchor barbs 156a are set parallel with anchor frame struts 152. Note also that anchor frame 150 may employ two attachment arms 158 at each implant facing crown, wherein some arms are provided with saw teeth 164 and some without. In another embodiment, the plane of the anchor barb direction can be offset such that it is in the axial direction of the flow of the blood within the IVC or any increment in between corresponding to axial direction over the indicated sizing range for the RC-WVM implant. FIG. 22C depicts an anchor barb 156a which in its final shape state lies parallel to the strut 150a which it is attached to, but is shape set such that its pointed tip is out of plane defined through the strut and parallel barb, that is out of the plane of the page as shown in FIG. 22C. This out of plane protrusion facilitates the anchor engaging with the vessel wall, preventing migration. The deployed configuration of this anchor is shown in FIG. 22A, with the anchor parallel to the strut 150a and therefore at an angle to the direction of blood flow in the vessel.

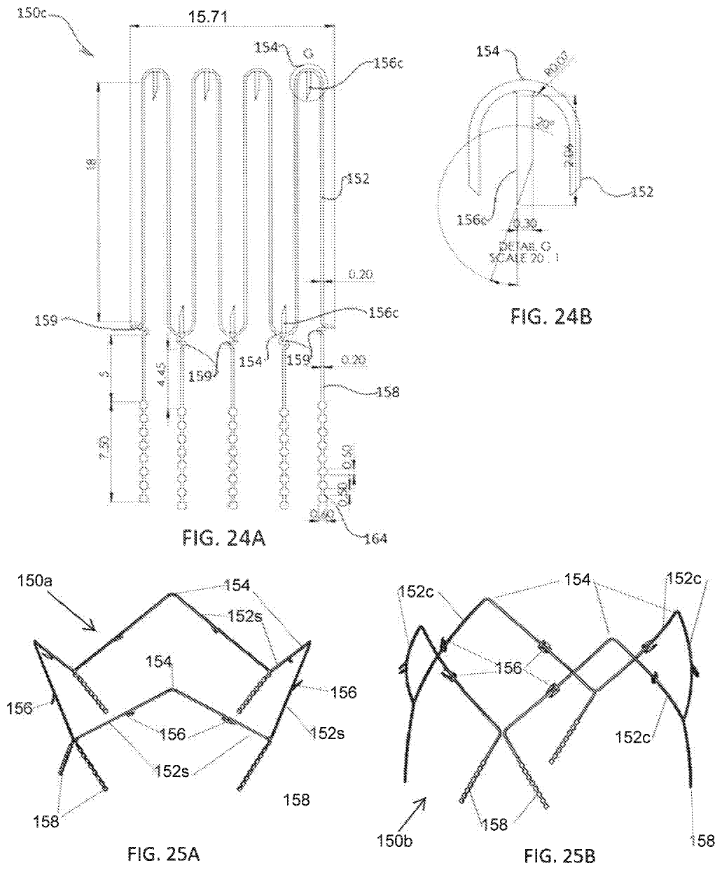

In another example, as shown in FIGS. 23A, 23B and 23C, axially facing anchor barbs 156b are positioned such that when anchor frame 150b is deployed within a vessel, anchor barbs 156b run parallel (or close to parallel) to the vessel direction and to the flow within the vessel. In a further embodiment, shown in FIGS. 24A and 24B, anchor barbs 156c of anchor frame 150c are located at crowns 154 of the anchor frame and shape set outwardly so as to engage the vessel wall. FIGS. 24A and 24B also provide an example of possible, approximate dimensions for an embodiment of an anchor frame. FIG. 23C depicts an anchor barb 156b which in its final shape state lies at an angle to the strut 150b which it is attached to, and is shape set such that its pointed tip is also out of the plane defined between the anchor barb and the strut to which it is attached. This out of plane protrusion in two axes, facilitates the anchor engaging with the vessel wall in a more optimal, more axial orientation, potentially providing increased migration resistance. The deployed configuration of this anchor is shown in FIG. 23A, with the anchor at an angle to the strut 150b and therefore generally parallel to direction of blood flow in the vessel. This final position of the anchor tip, out of plane from the strut in two axes can also be seen in FIG. 25A.

FIG. 25A depicts an anchor frame embodiment 150a, which is formed with straight strut sections 152s between crown sections 154. Straight strut sections 152s can provide an advantage of the strut section always being in contact with the vessel wall over its entire length, irrespective of the size of vessel into which it is deployed. When the frame is formed, for example, by laser cutting the construct from a nitinol tube, the straight configuration of straight strut sections 152s can be achieved by shape-setting the strut sections to maintain the desired straight configuration. FIG. 25B shows an alternative anchor frame embodiment 150b, which is formed around the surface of a cylindrical shape setting mandrel resulting in curved strut sections 152c. Curved strut sections 152c can provide the advantage of increasing the local force urging anchor barbs 156 (shown as double barbs) into the vessel wall for fixation, but may be associated with a disadvantage of the crowns not being in contact with the vessel wall, especially when the device is implanted in a small vessel.

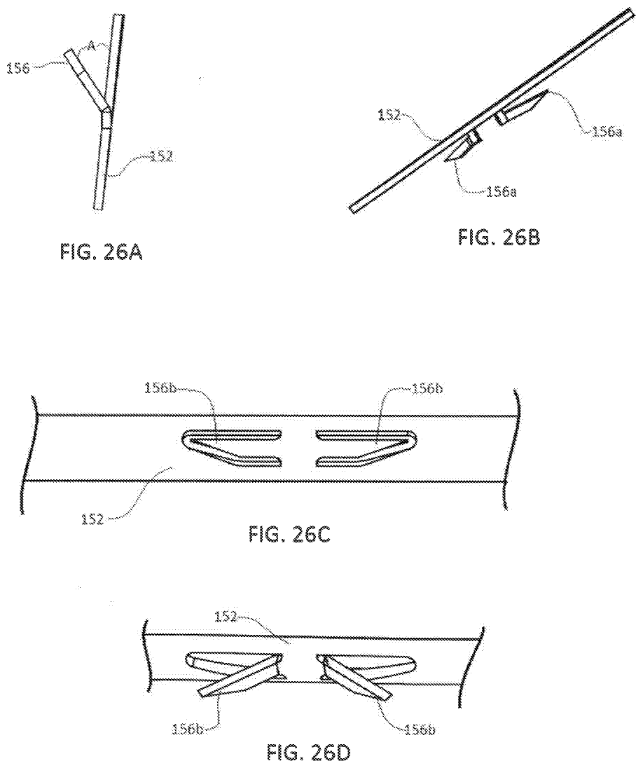

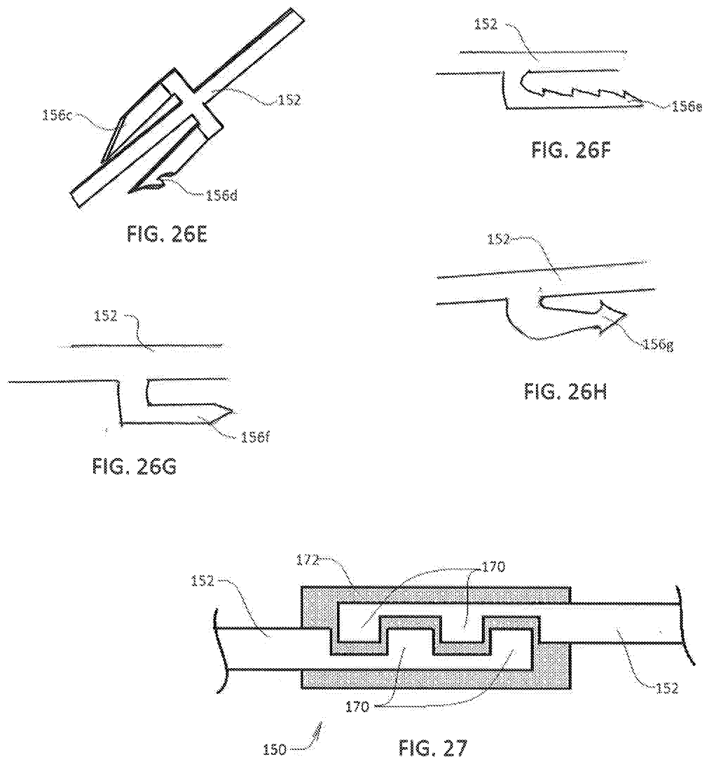

Various orientations and configurations of anchor barbs 156 may be provided in different embodiments as illustrated in FIGS. 26A-26G. For example, as shown in FIG. 26A, anchor barb 156 may extend outwardly at the center of each strut 152 of anchor frame 150 at an angle (A) between about 10° and 90°. Anchor barbs 156 may alternately face in either or both the caudal or cranial direction in the plane of the shape set strut 152 or extend out of that plane. In another embodiment, as shown in FIG. 26B, there may be multiple anchor barbs 156a on each strut 152 facing each direction. Multiple anchor barbs 156a as shown in FIG. 26B are located on one side of strut 152 facing in opposite directions, whereas in FIG. 26E, anchor barbs are on opposite sides of the strut, facing in the same direction. In another embodiment, shown in FIGS. 26C and 26D, anchor barbs 156b are contained within the thickness of strut 152, as opposed to being located on the side of the strut as shown, for example, in FIGS. 26A and 26B. The anchor barb configuration shown in FIGS. 26C-D may be formed in a similar manner to anchor barbs 50s as shown in FIGS. 14A-C and described above.

In other embodiments, examples of which are shown in FIGS. 26E-H, anchor barbs 156 may have overall shapes and/or points of different configurations, which may aid insertion and retention of the anchor barb within the vessel wall in various clinical situations. FIG. 26E illustrates single pronged barb 156c and fish hook barb 156d positioned on opposite sides of strut 152, facing in the same direction. FIGS. 26F, 26G and 26H show further examples of anchor barb designs, in this case saw-teeth barb 156e, double edged barb 156f, and double sided, hooked barb 156g, respectively. These barbs also can be located on the side of the anchor frame strut and also within the thickness of the strut as previously described

As described above, it may be desirable to configure anchor frame 150 so that it does not form a coil that could interfere with the RC-WVM implant signal. One solution, as described above is split 166. In other embodiments, for example where other design considerations may make a discontinuous structure less preferable such that anchor frame wire is mechanically and electrically joined (e.g. a crimped joint), the terminations of the wire ends where joined and in contact with each other may be electrically insulated so as to not form coil capable of coupling with a magnetic field. An example of such insulation is a polymer coating. In other embodiments, for example, where the anchor frame may be formed of nitinol laser cut tubing, for which a mechanical joint or bond may be required, the terminations of the nitinol frame can be physically and electrically separated by use of a non-conducting bonding agent such as a polymer, epoxy or ceramic material. FIG. 27 illustrates such a non-conducting joint in cross-section. In this example, ends 170 of anchor frame 150 have interlocking portions which may be bonded with non-conducting bonding agent 172, which also surrounds the joint for increased strength.

As previously discussed, the radial force exerted by the RC-WVM implant should be such that the sensor section moves with the natural motion of the IVC as it expands and contracts due to changes in fluid volume. Anchor frame 150 is configured to exert an outward radial force that is sufficient to ensure engagement of anchor barbs 156 into the vessel wall to help prevent migration along the vessel without interference with motion and electrical performance of the RC-WVM sensor section. Thus, the radial force exerted by anchor frame 150 typically may be equal to or higher than that exerted by the sensor section of the RC-WVM implant, so as to provide migration resistance while substantially isolated by isolation section 159 from the lower radial force sensor section, which, is configured to permit natural expansion and contraction of the IVC in response to varying fluid status.

Isolation section 159 allows attachment between the sensor section and anchor frame, but also permits the sensor section and anchor frame to act independently of each other. Thus, the RC-WVM sensor section can contract and expand at the monitoring location within the vessel independently of anchor frame expansion and contraction at the anchoring location in the vessel. One design consideration in selecting the configuration of the anchor frame is that the radial force exerted by the anchor frame should be sufficient to prevent migration of the RC-WVM implant, but low enough so as to not stent or prop open the vessel.

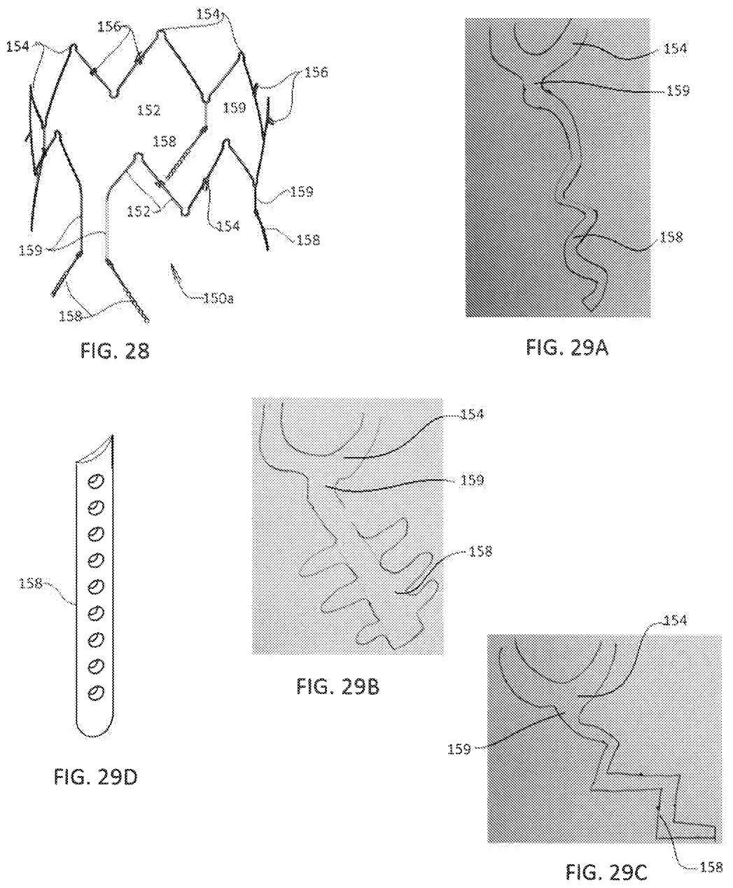

FIG. 28 illustrates one example of how the radial force of anchor frame 150 can be adjusted or modified to control the radial force exerted by altering the configuration, via changes in shape set diameter, strut width, strut thickness, strut shape, crown diameter, number of crowns, strut length, material properties, distance between the sensor section and anchor frame, and overall length. Another alternative to increase the fixation of the RC-WVM implant is to provide anchor frames on both ends of the sensor section, as shown in the example of FIG. 21. FIG. 28 shows an alternative anchor frame 150a with relatively short strut 152 lengths, more crowns 154 (here 16 crowns instead of 8 as in earlier embodiments), and smaller crown diameters. Isolation sections 159 are also longer so that the distance between the anchor frame and sensor section is increased.

The configuration of anchor frame 150a in FIG. 28 is selected for appropriate radial force while minimizing areas of high strain concentration that could lead to reduced fatigue life. Factors that affect the amount of radial force that can be exerted by the anchor frame without undue effect on the sensor section include the distance between anchor barbs 156 and the sensor section, which can be adjusted based on the position of the anchor barbs on strut 152 and/or by the length of isolation section 159 that also assists with isolation. In addition to varying the length of isolation section 159, other adjustments include varying the thickness and/or straight versus curved sections. For example, a straight anchor isolation section 159 is shown in FIG. 28, and in another example, a curved or s-shaped anchor isolation section 159 is shown FIG. 24A.

In another alternative embodiment, the anchor frame may be configured so as to intentionally fracture and self-separate from the sensor section over time. In this embodiment, connection points between the anchor frame and sensor section, for example in isolation section 159, are designed to deliberately fracture. The purpose of the deliberate fracture is to completely isolate the anchor frame from the sensor section after fracture. In such an embodiment, the anchor frame would secure the RC-WVM implant against migration when first deployed in the vessel. Over time, as the sensor section embeds into the tissue, the risk of migration diminishes. As a result, the anchor frame's function is no longer required. This embodiment allows for disconnection of the anchor frame from the device once it is no longer required without the need for surgical intervention.

The material and design of the isolation sections 159 may be selected to provide for different time periods for fracture to occur. For example, the geometry, design, movement and material of the sensor section, isolation section and anchor frame can be tuned for a fatigue induced fracture to occur after/within a given time due to fatigue. Alternatively, fracture can be induced by external means. For example ultra sound/RF may be used to induce fracture by breaking down the material or bond between the anchor frame and sensor section at a pre-set frequency or energy. In a further alternative embodiment, chemically induced fracture of isolation sections 159 may be achieved with, for example, a biodegradable polymer such as PLA, PCL, PLGA, PLG or other as the bond/connection between the anchor frame and RC-WVM implant frame. Chemically induced fracture takes advantage of the material properties of biodegradable polymers, which can degrade at controlled rates including such as of pH, temperature, microorganisms present, and water etc.

In another alternative embodiment, anchor frame 150 may be made of a bioabsorbable/biodegradable material such as commonly used for bioabsorbable stents. Similar to other embodiments of the anchor frame, the purpose of a bioabsorbable anchor frame is to help prevent migration. Once again, as the sensor section embeds into the tissue over time, the risk of migration diminishes. As a result, the anchor frame's function is no longer required. The material and design of a bioabsorbable anchor frame may be selected for different time periods for absorption.

The foregoing has been a detailed description of illustrative embodiments of the invention. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1.-9. (canceled)

10. A vascular implant, comprising a resilient, concentric zig-zag structure formed by multiple straight strut sections joined to one another by rounded crown sections, wherein:
said straight strut sections and crown sections are configured to position each entire straight strut section and each crown section in contact with the vascular lumen wall when deployed in a vascular lumen, whereby a plurality of vascular lumen diameters may be accommodated with a single size anchor frame.

11. The vascular implant of claim 10, further comprising an anchor isolation section disposed between each attachment section and each said crown section, the anchor isolation section being configured to allow independent motion between the anchor section and attachment section.

12. The vascular implant of claim 10, wherein said frame is electrically discontinuous with a nonconducting gap formed in said zig-zag structure.

13. The vascular implant of claim 12, wherein said nonconducting gap comprises one said crown section forming a split crown with two crown section pieces separated by a gap and an attachment section connected to each crown section piece.

14. The vascular implant of claim 10, wherein each tissue-engaging barb is disposed on a strut section at an angle with respect to the strut section such that, when the anchor frame is deployed within a vascular lumen in contact with the lumen wall, said barb is positioned generally parallel to a direction of blood flow in the vascular lumen.

15. The vascular implant of claim 10, wherein, each attachment section elongate member has a structure defining space for a bonding agent to enter and attach to the vascular implant.

16. The vascular implant of claim 10, wherein said attachment section elongate members comprise an alternating ridge and groove-like structure with the groove-like areas providing said spaces for the bonding agent to attach to the implant between the ridge-like structures.

17. The vascular implant of claim 10, wherein said attachment section elongate members include a series of holes formed defining said spaces.

18. The vascular implant of claim 10, wherein said attachment section comprises a spiral member with an inner diameter configured to be received over an outer diameter of an implant structure.

19. The vascular implant of claim 10, wherein said zig-zag structure has two ends, with said crown sections each disposed at one of the two ends with the strut sections in between; and at least a plurality of said straight strut sections form anchor sections with at least one tissue-engaging barb.

20. The vascular implant of claim 19, wherein:
at least one attachment section is connected to each crown section on at least one said end of said structure;
each attachment section comprises an elongate member defining spaces between structure of the attachment section for a bonding agent to enter and attach to the vascular implant.

21.-25. (canceled)

26. A vascular implant adapted to be deployed and implanted in a patient vasculature and positioned at a location in a vascular lumen in contact with the lumen wall, said implant comprising an anchor frame attached to a vascular device, the anchor frame comprising a resilient, concentric zig-zag structure formed by multiple strut sections joined to one another by rounded crown sections, wherein:
said concentric zig-zag structure has two ends, with said crown sections each disposed at one of the two ends with the strut sections in between;
at least a plurality of said strut sections form anchor sections with at least one tissue-engaging barb;
at least one attachment section is connected to each crown section on at least one said end of said structure;
each attachment section comprises an elongate member defining spaces between structure of said elongate member; and
a bonding agent is disposed within said spaces of each attachment section elongate member, bonding the attachment section to the vascular implant, with each attachment section attached to a separate section of said vascular device.

27. The vascular implant of claim 26, wherein said bonding agent comprises reflow material melted into the spaces defined by the attachment section elongate members to secure the attachment sections to the vascular device.

28. The vascular implant of claim 26, wherein:
the vascular device comprises a resilient sensor construct configured to dimensionally expand and contract with natural movement of the lumen wall;
an electrical property of the resilient sensor construct changes in a known relationship to the dimensional expansion and contraction thereof; and
said resilient sensor construct produces a wireless signal indicative of said electrical property, said signal being readable wirelessly outside said vascular lumen to determine a dimension of the vascular lumen.

29. The vascular implant of claim 28, wherein the resilient sensor construct comprises a resilient, concentric zig-zag structure formed by multiple straight strut sections joined to one another by rounded crown sections, with said straight sections configured to permit apposition of each entire straight strut section and each crown section against the vascular lumen wall for a plurality of vascular lumen diameters with a single size resilient sensor construct;

30. The vascular implant of claim 29, wherein:
said resilient sensor construct is configured and dimensioned to engage and substantially permanently implant itself on or in the lumen wall;
said resilient sensor construct has a variable inductance correlated to its dimensional expansion and contraction along at least one dimension; and
said resilient sensor construct produces, when energized by an energy source directed at said construct, a signal readable wirelessly outside the patient's body indicative of the value of said at least one dimension, whereby a dimension of the vascular lumen may be determined.

31. The vascular implant of claim 30, wherein said resilient sensor construct comprises a coil configured to engage at least two opposed points on the vascular lumen wall, said coil having an inductance that varies based on the distance between said two opposed points on said coil corresponding a distance between said points on the lumen wall.

32.-49. (canceled)

50. An anchor frame for a vascular implant, comprising:
a resilient, concentric zig-zag structure formed by multiple strut sections joined at acute angles to one another by rounded crown sections;
at least one tissue-engaging barb disposed in a plurality of said strut section;
means for attaching the zig-zag structure to a vascular implant; and
a nonconducting gap formed in said zig-zag structure such that the anchor frame is electrically discontinuous.

51. The anchor frame of claim 50, wherein:
the resilient, concentric zig-zag structure has two ends, with said crown sections each disposed at one of the two ends with the strut sections in between;
said means for attaching comprises at least one attachment section is connected to plural crown sections on at least one said end of said structure; and
each attachment section comprises an elongate member defining spaces between structure of the attachment section for a bonding agent to enter and attach to the vascular implant.

52. The anchor frame of claim 51, wherein said means for attaching includes an anchor isolation means for allowing independent motion between said resilient, concentric zig-zag structure and an implant attached thereto.

53. The anchor frame of claim 50, wherein said nonconducting gap comprises one said crown section forming a split crown with two crown section pieces separated by a gap and an attachment section connected to each crown section piece.

54. The anchor frame of claim 50, wherein, when deployed in a vascular lumen the anchor frame expands to contact the lumen wall, and said strut sections are straight to permit apposition of each entire strut section and each crown section against the vascular lumen wall for a plurality of vascular lumen diameters with a single size anchor frame.

55.-62. (canceled)
